SEQUENCE LISTING (2 sequences)

SEQ ID NO: 1 - DNA, 28 bp, synthetic construct
  source 1..28: mol_type = other DNA
gcacttaagaattgcaattcttaagtgc

SEQ ID NO: 2 - DNA, 28 bp, synthetic construct
  source 1..28: mol_type = other DNA
gcacttaagaattgcaattcttaagtgc

(19) United States
(12) Patent Application Publication
(10) Pub. No.: US 2024/0156764 A1
(43) Pub. Date: May 16, 2024

(54) INHIBITION OF URACIL DNA GLYCOSYLASE IN THE OPEN CONFORMATION

(71) Applicant: CASE WESTERN RESERVE UNIVERSITY, Cleveland, OH (US)

(72) Inventor: Stanton Gerson, Hunting Valley, OH (US)

(21) Appl. No.: 18/176,877

(22) Filed: Mar. 1, 2023

Related U.S. Application Data
(60) Provisional application No. 63/315,181, filed on Mar. 1, 2022.

(51) Int. Cl. A61K 31/194; A61K 31/506; A61K 31/519; A61P 35/00

(52) U.S. Cl. CPC A61K 31/194; A61K 31/506; A61K 31/519; A61P 35/00

(57) ABSTRACT

A method of treating cancer in a subject in need thereof includes administering to the subject an agent that inhibits uracil-DNA glycosylase wherein the agent binds to UDG such that the UDG is maintained in a destabilized, open precatalytic glycosylase conformation that prevents active site closing for functional DNA binding and nuclease flipping needed to excise damaged bases binding in DNA.

### RELATED APPLICATION

This application claims priority from U.S. Provisional Application No. 63/315,181, filed Mar. 1, 2022, the subject matter of which is incorporated herein by reference in its entirety.

### GOVERNMENT FUNDING

This invention was made with government support under CA092584, CA220430, and CA043703 awarded by The National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Jul. 10, 2023, is named CWR-030181US-ORD st.26 and is 2,904 bytes in size.

### BACKGROUND

Base excision repair (BER) protects DNA from intrinsic spontaneous base modification, such as cytosine deamination plus replication errors, endogenous oxygen radicals, extrinsic chemical mutagens, and alkylation as well as misincorporation due to nucleotide pool imbalance and incorporation of abnormal bases introduced as cancer chemotherapeutic agents. Pyrimidine analogs, such as 5-FdU, are metabolized to nucleotides and incorporated directly into DNA while antifolate drugs such as PEM blocks the conversion of UMP to TMP by thymidylate synthase, causing nucleotide pool imbalance and resulting in the incorporation of uridine during DNA synthesis.

BER explicitly detects, removes, and processes single base lesions in the DNA. Once a lesion is formed in DNA (single strand or double helix), it is detected by a DNA glycosylase, the first step of BER, which coordinates the removal of the damaged base by severing the N-glycosidic bond between the base and deoxyribose, forming an apurinic/apyrimidinic (AP) site. Eleven known human DNA glycosylases detect and process a wide range of DNA lesions, including deaminated, alkylated bases, and oxidized bases. Following excision of the damaged bases, AP endonuclease is recruited to the AP site, where it flips the AP site and cleaves the sugar backbone of the DNA, further activating different downstream BER proteins from either the small patch or long patch sub-pathway to ultimately repair the DNA. DNA glycosylases are small DNA binding proteins that form monomers, and they do not require co-factors to function with the exception of some that contain 4Fe-4S clusters such as MutY and endonuclease III, and some can also cleave the AP site.

Uracil-DNA glycosylase (UDG) was the first DNA glycosylase discovered. UDG removes with high-efficiency uracil as well as the synthetic 5-FU, 5-hydroxyuracil, alloxan, and isodialuric acid, but at significantly lower rates. Furthermore, accumulating evidence suggests that blocking UDG with a specific inhibitor could have therapeutic benefits by improving the anti-cancer effect of existing chemotherapeutic agents, such as Pemetrexed (PEM), which in previous publications have shown to promote uracil incorporation through inhibition of thymidylate synthase (TS). Similarly, 5-FdU also inhibits TS or the drug is metabolized to 5-FdUTP and is incorporated along with uracil due to inhibition of TS. In fact, shRNA/siRNA-mediated abrogation of UDG expression decreased the viability of human cancer cell lines treated with either PEM or 5-FdU. These data imply that UDG-dependent repair activity can dictate the sensitivity of cancer cells to chemotherapeutic drugs like PEM and 5-FdU, which are dependent on the incorporation of uracil and 5-FU into DNA.

### SUMMARY

Embodiments described herein relate to a method of treating cancer in a subject in need thereof. The method includes administering to the subject a therapeutically effective amount of an agent that inhibits uracil-DNA glycosylase (UDG). The agent or UDG inhibitor (UDGi) binds to UDG such that the UDG is maintained in a destabilized, open precatalytic glycosylase conformation that prevents active site closing for functional DNA binding and nuclease flipping needed to excise damaged bases binding in DNA.

In some embodiments, the UDGi is administered to a subject in combination with at least one of a folate antimetabolite or pyrimidine analog.

In some embodiments, the UDGi has a Kd of <700 nM and an IC₅₀ of less than <700 nM for UDG.

In other embodiments, the UDGi binds free UDG prior to DNA binding.

In some embodiments, the UDGi promotes destabilization of UDG.

In some embodiments, the UDGi has a non-uracil chemotype.

In some embodiments, the UDGi is aurintricarboxylic acid (ATA), an analog, derivative, or chemotype thereof, or a pharmaceutically acceptable salt, tautomer, or solvate thereof.

In other embodiments, the folate antimetabolite or pyrimidine analog includes at least one of pemetrexed, 5-FdU, or 5-FU.

### BRIEF DESCRIPTION OF THE DRAWINGS

FIGS. 1(A-D) illustrate high-Throughput Small-Molecule Screening for UDG Inhibitors. A: 60 min reaction time-course experiment was performed to measure human UDG enzymatic activity in the presence of 10 nM UDG (red line) or absence of UDG (black line) uracil DNA hairpin (25 nM) only control. Each data point is the average of 12 reaction replicates from a 384-well plate. Error bars represent the standard deviation from the mean. B: Two independent screens using a plate of 320 compounds were plotted on the same graph with the trend line showing a positive correlation between the two runs with an R2 value of 0.711 using y=mx+b. C: A total high-throughput screen with 3115 bioactive compounds was done. Normalized percent activity of UDG-mediated unquenching of fluorescence DNA hairpin is shown. See methods for further detail regarding the equation that was used to normalize the percent activity of UDG. Control condition denotes as RFU+UDG AVG (DMSO controls without compound); the average RFU value was computed from a set of 32 wells per screening plate. Control condition denotes RFU-UDG AVG (DNA alone no protein); the average relative fluorescence unit (RFU) value was computed using a set of 32 replicate wells per screening plate. D: A dose-response curve showing the normalized average % inhibition of UDG-mediated un-quenching of fluorescence from the DNA hairpin over nine different concentrations of ATA, starting with the highest dose of 12.5 μM, then serially diluted to 3.96 μM, 1.25 μM, 0.396 μM, 0.125 μM, 0.0396 μM, 0.01255 μM, 0.003972 μM, 0.001257 μM, and 0.0003978 μM. Each point at each concentration represents a normalized average percent inhibition from three replicate wells from three different 384-well plates. The IC50 value was generated using Prism software version 8.4.3, non-linear fit, [inhibitor] vs. response (three parameters, R2=0.99). Y error bars represent the standard deviation from the mean. The average RFU+UDG AVG (UDG+DMSO control without compound) value was computed from a set of 18 wells per screening plate. The average RFU-UDG AVG (uracil DNA hairpin alone) was computed using a set of 9 wells per screening plate.

FIGS. 2(A-E) illustrates ATA binds and cocrystallizes with the human UDG. A: Crystal Structure and density map of UDG-ATA complex at 1.8 Å resolution and the 2D interacting map. Density map at 0.7 sigmas for ATA bound to UDG at 1.8 Å resolution (PDB ID: 6VBA). The ligand is bound to the active site and shows direct stacking with TYR147 and HIS268. Two water molecules are also directly interacting with the ligand. B: 2D interacting map generated with Maestro molecular viewer shows in detail key residues involved in the UDG-ATA binding complex. C: a cartoon of the open unbound UDG catalytically incompetent conformation. D: a cartoon of the closed bound UDG conformation. E: a cartoon of ATA bound open unbound UDG.

FIGS. 3(A-C) illustrate ATA directly binds and destabilizes the human UDG protein. A: ATA binding to UDG measured by MST. ATA (30 nM to 500 μM) was titrated into a fixed concentration of labeled UDG (100 nM). The data for thermophoresis was recorded at 20° C. using the blue LED at 20% and IR-Laser at 40%. The isotherm derived from the raw data and fitted according to the law of mass action. B: UDG Protein thermal shift with sybro orange dye was performed with a total volume of 20 μL per reaction included 5 μL of a buffer from the kit (Applied biosystems cat #4461335), 10 μL of storage UDG buffer or UDG protein (protein stock 0.8 μg/μL), 1 μL of DMSO or ATA compound (stock: 10,000 μM, 2000 μM, or 400 μM), Reaction plate was incubated in the dark on ice for 20 min. Then 2 μL of 10× Sypro dye and 2 μL of ddH₂0 (Applied biosystems cat #4461141) were added. Monitoring of fluorescence took place for 2 hr (Bioraid CFX96, Hex filter, 25-97° C., 1.6° C./2 min). Negative (−) derivative RFU raw data and plots from the machine were obtained and analyzed. The plot shows negative −dRFU/dt over temperature, 16 μM of UDG protein (minus ATA), orange curve), 20 μM ATA+16 μM of UDG (blue curve), 100 μM ATA+16 μM UDG (light blue curve) and 500 μM+16 μM of UDG protein (red curve). Melting temperature (Tₘ) value was taken directly from the midpoint on the −dRFU/dt vs. temperature graph. Each point on the graph represents an average of three data sets. Y error bars represent the standard deviation. C: Monitoring the thermal stability of UDG in the presence of titrating concentrations of ATA through intrinsic tryptophan fluorescence. UDG with 0 μM ATA is at 53.5° C. shown by a dotted line on the first derivative peak corresponding to the red lines on the 330 nm panel.

FIGS. 4(A-D) illustrate ATA activity on purified human UDG and 8oxoguanine DNA glycosylases. A: ATA diminishes UDG-mediated DNA cutting activity in a dose-dependent manner using single uracil:adenine base-pair DNA cleavage assay. 20% polyacrylamide nucleic acid urea gel electrophoresis was used to resolve the DNA bands at room temperature, 200 v for 50 min. Analysis and quantification of DNA cleavage were done as follows. A background subtraction step was included using the 23mer from the DNA only lane. Dose-response plot showing relative average percent 23-mer cleaved single-stranded DNA band divided by the total amount of DNA product bands (23mer+40mer) over different ATA concentrations. The lane with UDG (500 pM)+DNA (20 nM) without any compound (DMSO only) was set as 100% cleavage and was used as the denominator for subsequent points with ATA (12.5 μM, 4.2 μM, 1.3 μM, 0.46 μM). Lane 9 serves as a control using a thymidine:adenine DNA basepair in the presence of UDG does not show DNA cleavage. B: Points on the plot were averaged from four different gels. The IC₅₀ value was generated using Prism software version 8.4.3, non-linear fit, [inhibitor] vs. response (three parameters, R2=0.99) averaging points from four different gels. Y error bars represent the standard deviation from the mean. C: ATA displayed a lesser inhibitory effect on purified human OGG1 protein (10 nM). A single oxo-guanine: cytosine base pair DNA with a Cy3 fluorescent tag on the 3′ end was used (10 nM). D: The IC50 value was generated using Prism software version 8.4.3, non-linear fit, [inhibitor] vs. response (three parameters, R2=0.98) averaging points from three different gels.

FIGS. 5(A-C) illustrate evaluations of the inhibitory effect of ATA on cell-free protein extracts from DLD1 human colon cancer cell line. A: human cancer lysates were extracted from DLD1 human colon cancer cell lines. UDG containing DLD1 cells (wild type and sh scramble), DLD1 cell line with a sh-knockdown UDG, and DLD1 cell line with a CRISPR-knockout UDG were treated with ATA. Reactions were done in 96 well plate format using 20 μg of lysate pre-incubated with either vehicle DMSO (gel lanes 1,3,5&7) or 12.5 μM of ATA (gel lanes 2,4,6&8) for 20 min at 37° C. and then a final 30 nM of fluorescently-labeled TAMRA DNA duplex oligo (uracil:adenine bp or thymidine:adenine bp) was added to the reaction for another 20 min incubation at 37° C. Lane 9 contained uracil DNA only condition (without cell lysate and without the ATA compound). Lane 10 contained uracil DNA+12.5 μM of ATA in the absence of any cell lysate. B: 50.0 μM of ATA was tested in gel lane 2,4,6&8. 20% polyacrylamide nucleic acid urea gel electrophoresis was used to resolve the DNA bands at room temperature, 200 v for 50 min. DNA products were visualized using a Typhoon Trio+Variable Mode Imager (Amersham Biosciences) with excitation and emission wavelengths set to 532 nm and 585 nm respectively, PMT set to 400, and pixel size resolution set to 100 μm. C: IC50 estimation of ATA inhibition effect on DLD1 UDG containing cell lysate. Human DLD1 colon cancer lysate was treated over a range of ATA (0-50 μM) for 20 min at 37° C. Either uracil DNA (U:A bp) or thymidine DNA (T:A bp) was added to initiate the reaction and incubated for additional 20 min at 37° C. 20% polyacrylamide nucleic acid urea gel electrophoresis was used to resolve the DNA bands. Gel data were analyzed using ImageJ software. DNA bands were corrected using a constant background band intensity subtraction step. Then, percent cleavage was calculated by using fluorescence intensity of the cleaved 23-mer DNA strand divided by the sum of the fluorescence bands (23mer+40mer DNA).

FIG. 6(A-B) illustrate measurement of ATA in vivo using a host-cell reactivation assay. A: MCF-7 cells were seeded at 50,000 cells per well in a 12-well plate. B: HAP cells (wild type or UNG gene knockout) were seeded at 75,000 cells per well in a 6-well plate. Cells were allowed to adhere overnight and then cells in duplicate wells were treated with DMSO or ATA (2.5, 5, or 10 μM) for 24 hr (refer to methodology). After 24 hours, cells were dissociated by trypsinization and analyzed by flow cytometry using an Atune NxT flow cytometer. Gating and compensation were determined by transfection of single-color controls. Reporter expression was calculated for each dose of ATA as previously described in publications and normalized to DMSO treatment.

FIGS. 7(A-B) illustrate measurement of ATA inhibition on uracil, 8oxoG:C, A:8oxoG:C, Hypoxanthine:T and T:G repairs in MCF-7 cells using a host-cell reactivation assay with doses. Panel A: 1.25 μM and panel B: 2.5 μM. Reporter expression was calculated for each dose of ATA as described in prior publications and normalized to DMSO treatment.

FIGS. 8(A-H) illustrate aurintricarboxylic acid inhibits purified human UDG-mediated unquenching of multiple uracil DNA hairpin over time without damaging DNA fluorescence in vitro (A-F): human UDG activity time course with varying DNA concentrations, ATA while fixing protein concentration. A: 600 nM of uDNA hairpin, B: 400 nM of uDNA hairpin was incubated with 50 pM of UDG, C: 266 nM of uracil DNA hairpin D: 178 nM of uDNA hairpin and E: 118 nM uDNA hairpin was incubated with subnanomolar UDG (final 50 pM) and the addition of either DMSO or a fixed dose of ATA (final 500 nM) for 4 hr. uracil DNA hairpin alone (−UDG,−ATA) curve at different concentration was included in each group over the same duration (A-E). RFU was monitored over time. Each point on plot represents an average of three replicates. Error bar represents standard deviation from the mean. F: Human UDG activity was monitored over a time course with varying inhibitor ATA concentrations while fixing protein and DNA levels. 50 pM of hUDG was incubated with 600 nM u-DNA hairpin substrate with either DMSO or ATA (0-500 nM) over a 5 hr time course at room temperature. G: In the absence of UDG protein, 600 nM uracil DNA hairpin+DMSO only reaction and uracil DNA hairpin+ATA (final 500 nM) reaction were monitored over a 5 hr. time course at room temperature. Each point on the plot is the average of 4 wells (ATA condition). Each data point on the plot is the average of 3 wells (DMSO condition). Error bars represent standard deviation from the mean. H: uDNA hairpin melting over ATA concentration. UDG protein was not added to each reaction. Uracil DNA hairpin in buffer only condition and uracil DNA hairpin+ATA were monitored over increasing temperature. Fluorescently labeled FAM-uDNA hairpin was monitored over the melting of the DNA hairpin. Uracil DNA hairpin final concentration was 500 nM while ATA varied (0.12 μM, 0.25 μM, 0.49 μM, 0.98 μM, 1.9 μM, 3.9 μM, 7.8 μM and 15.6 μM). Each point represents an average of 4 experimental wells. Y Error bars represent the standard deviation from the mean.

FIGS. 9(A-I) illustrate summary of docking results of uracil, ATA and salicylic acid to UDG and diagram of protein-ligand interactions. A: PyMOL session of superposition of top uracil results docked to the four UDG structures analyzed in this study. All uracil results are superposed to the PDB structure 1SSP (cyan). Respectively, scarlet uracil docked to UDG from 1UGH structure, gray uracil docked to UDG from 1EMH structure, yellow structure docked to UDG from 1AKZ structure, and magenta uracil docked to UDG from 1SSP structure. B: 2D interaction map of uracil ring bound to UDG active site with specific hydrogen bond interactions and pi-pi stacking. C: PyMOL session of superposition of top ATA results docked to UDG from 1UGH structure. All ATA results are superposed to the PDB 1EMH (cyan). D: PyMOL session of superposition of top uracil results docked to the four UDG structures analyzed in this study. All uracil results are superposed to the PDB structure 1EMH (cyan). Represented as violet stick is the top ATA result docked to UDG from 1UGH structure shown also in (C) and represented as line the multiple top results population from the ATA docked to the other UDG structures. E: 2D interaction map of ATA bound to UDG active site from 1UGH with specific hydrogen bond interactions and pi-pi stacking. F: PyMOL session of superposition of best Salicylic acid result (magenta) docked to apo UDG from 1AKZ and the best ATA result (gray) docked to UDG from 1UGH. Both results superposed to 1EMH (cyan). G: Same superposition as F: with the addition of all results of salicylic acid docked to UDG from apo 1AKZ as magenta lines. H: 2D interaction map of Salicylic acid bound to UDG active site from 1AKZ with explicit hydrogen bond interactions and pi-pi stacking. I:Docking of ATA to UDG from 1SSP (UDG bound to dsUDNA). The figure shows the six best poses of ATA docked with Glide to active site of UDG, docking aim to residue GLN144. All six poses follow the dsDNA backbone due to close conformation of UDG active site.

FIGS. 10(A-D) illustrate UDG thermal shift. A: Uracil DNA hairpin stabilizes UDG protein. A series of negative dRFU/dt over temperature plots represent the UDG protein (final 16 μM) melt profile alone +DMSO in the absence of ATA (orange curve), with the addition of 20 μM ATA (blue curve), and with the addition of 100 μM ATA (light blue curve). Melting temperature (Tm) values were taken directly from lowest peak point on the negative dRFU/dt over temperature plots. B: (with uracil DNA): Parallel set of wells on the same plate included uracil-DNA harpin (final 1.0 mM). Each plate was incubated 60 min at room temperature before the monitoring of fluorescence took place about 2 hr (Biorad CFX96, Hex filter, 25° C.-97C, 1.6° C./2 min). Each point represents the average of three experimental wells. Y error bars represent standard deviation from the mean. C: Thymidine DNA hairpin does not change the melting temperature of UDG protein without the addition of thymine DNA, the plot of dRFU/dt over temperature shows the UDG protein (final 16 μM) melt profile only without ATA condition (orange curve), 20 μM ATA+16 μM of UDG (blue curve), and 100 μM ATA+16 μM UDG (light blue curve). Melting temperature (Tm) values were taken directly from lowest peak point from curves without manipulation. D: (with thymine DNA): Parallel set of wells on the same plate included additional thymine-DNA (final 1.0 mM). Each plate was incubated 60 min at room temperature before the monitoring of fluorescence took place about 2 hr (Biorad CFX96, Hex filter, 25° C.-97° C., increment of 1.6° C./2 min). Each point represents the average of two experimental wells. Y error bars represent standard deviation from the mean.

FIG. 11 illustrates UNG mRNA level in UDG containing and shKD UNG human DLD1 colon cancer cell lines. The sequence and details of lentiviral shRNA knockdown are available in prior publication.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994.

Unless indicated otherwise, the following terms have the following meanings when used herein and in the appended claims. Those terms that are not defined below or elsewhere in the specification shall have their art-recognized meaning.

As used in the specification and the appended claims, the singular forms “a,” “an” and “the” include plural referents unless the context clearly dictates otherwise. Thus, for example, reference to “a substituent” includes a single substituent as well as two or more substituents that may be the same or different, reference to “a compound” encompasses a combination or mixture of different compounds as well as a single compound, reference to “a pharmaceutically acceptable carrier” includes two or more such carriers as well as a single carrier, and the like.

The term “agent” and “drug” are used herein to mean chemical compounds, mixtures of chemical compounds, biological macromolecules, or extracts made from biological materials, such as bacteria, plants, fungi, or animal particularly mammalian) cells or tissues that are suspected of having therapeutic properties. The agent or drug may be purified, substantially purified, or partially purified.

The term “antimetabolite” is used herein to mean a chemotherapeutic with a similar structure to a substance (a metabolite e.g., nucleoside) required for normal biochemical reactions, yet different enough to interfere with the normal functions of cells, including cell division.

The term “antineoplastic” is used herein to mean a chemotherapeutic intended to inhibit or prevent the maturation and proliferation of neoplasms (tumors) that may become malignant, by targeting the DNA.

The terms “neoplastic cell”, “cancer cell” or “tumor cell” refer to cells that divide at an abnormal (i.e., increased) rate. A neoplastic cell or neoplasm (tumor) can be benign, potentially malignant, or malignant. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, meningoma, medulloblastoma, schwannoma and epidymoma

The term “reverses resistance” means that the use of a second agent in combination with a primary chemotherapeutic is able to produce a significant decrease in tumor volume at a level of statistical significance (e.g., p<0.05) when compared to tumor volume of untreated tumor in the circumstance where the primary chemotherapeutic alone is unable to produce a statistically significant decrease in tumor volume compared to tumor volume of untreated tumor. This generally applies to tumor volume measurements made at a time when the untreated tumor is growing log rhythmically.

The term “potentiate” as used herein means to enhance or increase the beneficial activity or efficacy of the anticancer agent over that which would be expected from the anticancer agent alone or the potentiating agent alone.

The term “sensitize” as used herein means to alter cancer cells or tumor cells in a way that allows for more effective treatment of the associated neoplastic disease with an antimetabolite agent, an anticancer agent, or radiation therapy. In some embodiments, normal cells are not affected to an extent that causes the normal cells to be unduly injured by the antimetabolite, chemotherapy, or radiation therapy.

The term “subject” and “individual” are used herein interchangeably. They refer to a human or another mammal (e.g., primate, dog, cat, goat, horse, pig, mouse, rat, rabbit, and the like), that can be afflicted with cancer, but may or may not have the disease. In many embodiments, the subject is a human being.

The term “synergistic effect” as used herein means the combined effect of two or more anticancer agents or chemotherapy drugs can be greater than the sum of the separate effects of the anticancer agents or chemotherapy drugs alone.

The term “therapeutically effective amount” means the amount of the subject compound that will elicit a desired response, for example, a biological or medical response of a tissue, system, animal, or human that is sought, for example, by a researcher, veterinarian, medical doctor, or other clinician.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

Additionally, the salts of the compounds described herein, can exist in either hydrated or unhydrated (the anhydrous) form or as solvates with other solvent molecules.

Non-limiting examples of hydrates include monohydrates, dihydrates, etc. Nonlimiting examples of solvates include ethanol solvates, acetone solvates, etc.

The term “solvates” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water, the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The compounds and salts described herein can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Tautomerizations can be catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

The terms below, as used herein, have the following meanings, unless indicated otherwise:
“Amino” refers to the —NH₂ radical.
“Cyano” refers to the —CN radical.
“Halo” or “halogen” refers to bromo, chloro, fluoro or iodo radical.
“Hydroxy” or “hydroxyl” refers to the —OH radical.
“Imino” refers to the ═NH substituent.
“Nitro” refers to the —NO₂ radical.
“Oxo” refers to the ═O substituent.
“Thioxo” refers to the ═S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₈ alkyls but also includes C₆ alkyls. A C₁-C₁₀ alkyl includes all moieties described above for C₁-C₈ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alknyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “C_{w}-C_{z} acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₂-C₁₀ alkenyl, or C₂-C₁₀ alkynyl radical as defined above. Unless stated otherwise specifically in the specification, an alkyl carbonyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from phenyl (benzene), aceanthrylene, acenaphthylene, acephenanthrylene, anthracene, azulene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}—Rₑ where R_{b} is an alkylene group as defined above and Rₑ is one or more aryl radicals as defined above. Aralkyl radicals include, but are not limited to, benzyl, diphenylmethyl and the like. Unless stated otherwise specifically in the specification, an aralkyl group can be optionally substituted.

“Aralkenyl” or “arylalkenyl” refers to a radical of the formula —R_{b}—Rₑ where R_{b} is an alkenylene group as defined above and Rₑ is one or more aryl radicals as defined above. Unless stated otherwise specifically in the specification, an aralkenyl group can be optionally substituted.

“Aralkynyl” or “arylalkynyl” refers to a radical of the formula —R_{b}—Rₑ where R_{b} is an alkynylene group as defined above and Rₑ is one or more aryl radicals as defined above. Unless stated otherwise specifically in the specification, an aralkynyl group can be optionally substituted.

“Carbocyclyl,” “carbocyclic ring” or “carbocycle” refers to a ring structure, wherein the atoms which form the ring are each carbon. Carbocyclic rings can comprise from 3 to 20 carbon atoms in the ring. Carbocyclic rings include aryls and cycloalkyl. Cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused, bridged, or spiral ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused, bridged, or spiral ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused, bridged, or spiral ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkynyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused, bridged, and spiral ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, aziridinyl, oextanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, pyridine-one, and the like. The point of attachment of the heterocyclyl, heterocyclic ring, or heterocycle to the rest of the molecule by a single bond is through a ring member atom, which can be carbon or nitrogen. Unless stated otherwise specifically in the specification, a heterocyclyl group can be optionally substituted.

“Heterocyclylalkyl” refers to a radical of the formula —R_{b}—Rₑ where R_{b} is an alkylene group as defined above and Rₑ is a heterocyclyl radical as defined above. Unless stated otherwise specifically in the specification, a heterocyclylalkyl group can be optionally substituted.

“Heterocyclylalkenyl” refers to a radical of the formula —R_{b}—Rₑ where R_{b} is an alkenylene group as defined above and Rₑ is a heterocyclyl radical as defined above. Unless stated otherwise specifically in the specification, a heterocyclylalkenyl group can be optionally substituted.

“Heterocyclylalkynyl” refers to a radical of the formula —R_{b}—Rₑ where R_{b} is an alkynylene group as defined above and Rₑ is a heterocyclyl radical as defined above. Unless stated otherwise specifically in the specification, a heterocyclylalkynyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical one to thirteen carbon atoms and one to six heteroatoms selected from the group consisting of nitrogen, oxygen and sulfur, as the ring member. For purposes of this invention, the heteroaryl radical can be a monocyclic, bicyclic, tricyclic or tetracyclic ring system, which can include fused or bridged ring systems, wherein at least one ring containing a heteroatom ring member is aromatic. The nitrogen, carbon or sulfur atoms in the heteroaryl radical can be optionally oxidized and the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrazolopyridine, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}—R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}—R_{f} where R_{b} is an alkynylene chain as defined above and R_{f} is a heteroaryl radical as defined above. Unless stated otherwise specifically in the specification, a heteroarylalkynyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (e.g., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl, heteroarylalkyl, heteroarylalkenyl, heteroarylalkynyl, etc) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

The terms “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The terms “uracil DNA glycosylose” or “UDG” or “UNG” refer to a conserved DNA repair protein expressed in all types of human cells. It specifically removes uracil from DNA and protect cells from cytotoxicity and mutagenicity. Human UDG is encoded by the UNG gene. Alternative promoter usage and splicing of this gene produces two different isoforms: the mitochondrial UNG1 and the nuclear UNG2. Nuclear UDG (UNG2) is the predominant form in cells and represents greater than 90% of the total enzyme activity.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Embodiments described herein relate to compounds and methods of inhibiting uracil-DNA glycosylase in a cancer cell. UDG substrates, such as uracil and/or 2-fluoroadenine 9-β-Darbinofuranoside-triphosphate, can be incorporated into DNA of cancer cells by administering antimetabolite agents to the cancer cells. The UDG substrates can serve as a poor substrate for DNA replication enzymes, leading to the inhibition of DNA replication, chain termination, and loss of genome integrity. To maintain the genome integrity of the cancer cells, the cancer cells rapidly eliminate the UDG substrate from the DNA by base excision repair, which is initiated by the uracil-DNA-glycosylase (UDG or UNG) enzyme.

The UDG enzyme hydrolyzes the N-glycosidic bond between the UDG substrate (e.g., uracil residue) and the deoxyribose sugar of the DNA backbone, liberating the UDG substrate and generating an abasic site (e.g., an apurinic or apyrimidinic (AP) site). An apurinic or apyrimidinic (AP) site results from the loss of a purine or pyrimidine residue, respectively, from DNA (deoxyribonucleic acid). The AP site is further processed by a 5′-3′ endonuclease (AP endonuclease (APE)) that incises the phosphodiester bond on both sides of the damaged purine or pyrimidine base. The AP endonucleases can introduce chain breaks by cleaving the phosphodiester bonds at the AP sites.

We developed and applied an assay to screen UDG inhibitors (UDGis) resulting in identification and characterization of exemplary small molecule UDGis having a structure based on aurintricarboxylic acid (ATA), which are effective in inhibiting UDG base excision repair in human cancer cells. Our structural and computational results suggest a novel strategy to inhibit UDG by targeting the open catalytically inactive conformation that is both catalytically incompetent and not only less able to bind DNA but also less stable providing a possible dual means to reduce UDG activity in cancer cells. The UDGi bound structure and assays provide a chemical framework distinct from uracil substrate, which allows the closed DNA binding conformation.

We further found that UDGis based on ATA do not disrupt uracil-containing DNA in vitro, and directly bind to human UDG showing that UDGis based on ATA work by binding free UDG prior to its DNA binding. UDG recognition of uracil in DNA is a cooperative phenomenon involving multiple individual binding interactions of UDG with both DNA backbone and uracil nucleotide that enable the precise fit of the catalytically competent UDG-DNA substrate complex. Importantly, these interactions are synergistic and include conformational closing of both the DNA binding channel and the uracil binding pocket. By strategically interfering with some of these interactions and with the conformational closing required for specific uracil recognition and excision, agents based on ATA can constitute effective UDGis despite having multiple bound conformations, not resembling a transition state analog and not pushing open adjoining sites. Instead, ATA binds to the open active site, makes interactions with some key residues for uracil binding, and resists conformational closing that is the cooperative mechanism for uracil recognition and excision. This structurally-supported concept of binding the open state and blocking the conformational closing needed for substrate recognition explains our collective data on inhibition, binding, and lowering the UDG melting temperature. Importantly, novel synthesized molecules based upon ATA, chemically distinct from the natural uracil-DNA substrate, can similarly hold the UDG active site open to effectively block uracil recognition and removal.

Characterization of ATA as a UDGi can provide strategies for nucleic acid research and cancer. The small molecule need not compete with UDG binding to DNA. Instead, compounds that effectively hold UDG in the open (catalytically incompetent) and less stable conformation precluding damaged DNA substrate recognition and base removal may be equally or even more effective. This provides an enabling strategy for UDG as results from both hydrogen-deuterium exchange and computational docking show that the DNA binding area for UDG (and by implication other BER enzymes) is more significant than indicated in crystal structures suggesting it may be quite challenging to block with a small-molecule chemical inhibitor. Yet as a “door stopper” ATA does not need to compete with DNA; it only needs to block UDG closing and thus prevent the complementary DNA binding channel and the lock-and-key specific substrate recognition. In this analogy, the preclinical UDG inhibitor ATA need not have key-like specificity: instead, it needs to provide a non-uracil chemotype suitable for chemical optimization to improve affinity and specificity to the open UDG conformation. We therefore propose that the “door stopper” inhibitor strategy identified here by the UDG-ATA complex can be used as a potential general strategy to block excision repair enzymes for cancer biology.

Indeed, ATA effectively inhibits UDG-mediated cleavage of uracil-containing DNA in human DLD1 colon cancer cell extracts and in human cell lines. These observed reductions of UDG activity in human cancer cell lysates and cells suggests that an ATA-based inhibitor strategy may effectively block intracellular UDG activity. Binding data with the more soluble form of ATA (ATA NH4 salt) shows significant improvement in direct binding to the purified human UDG protein. The structural and docking results show that by binding the open UDG conformation ATA decrease stable DNA binding and blocks uracil flipping and excision.

Taken together, our findings identify ATA, derivatives, chemotypes, and analogues thereof that are able to inhibit UDG by holding the protein in the open conformation, like a door stopper, that can effectively inhibit UDG in cells and cell extracts despite the presence of large amounts of potential non-specific DNA binding.

Accordingly, in some embodiments, a method of treating cancer in a subject in need thereof includes administering to the subject a therapeutically effective amount of an agent or UDGi that binds to a UDG such that the UDG is maintained in a destabilized, open precatalytic glycosylase conformation that prevents active site closing for functional DNA binding and nuclease flipping needed to excise damaged bases binding in DNA and administering the selected agent to the cancer cells.

In some embodiments, the agent or UDG inhibitor (UDGi) has a Kd of <700 nM and an IC₅₀ of less than <700 nM for UDG.

In other embodiments, the UDGi binds free UDG prior to DNA binding.

In some embodiments, the UDGi promotes destabilization of UDG.

In some embodiments, the UDGi has a non-uracil chemotype.

In some embodiments, the UDGi is aurintricarboxylic acid (ATA), an analog, derivative, or chemotype thereof, or a pharmaceutically acceptable salt, tautomer, or solvate thereof. The term “aurintricarboxylic acid”, in the following also abbreviated with the term “ATA”, includes salts, tautomers, and solvates thereof unless otherwise indicated. In addition, ATA includes not only the monomeric aurintricarboxylic acid but also oligomeric and polymeric forms thereof, including rearrangement products containing the basic salicylic acid structure. Examples of the various forms of ATA are shown in Wang, P., et al., J. Org. Chem. 1992, 57, 3861-3866, which is incorporated by reference herein.

The basic structure of ATA is shown as formula I.

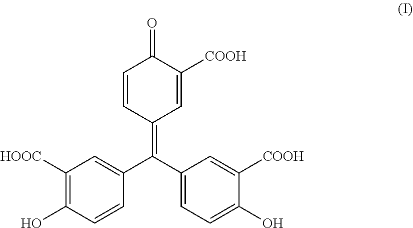 [SMILES: O=C(O)C1=CC(=C(c2ccc(O)c(C(=O)O)c2)c2ccc(O)c(C(=O)O)c2)C=CC1=O]

or a pharmaceutically acceptable salt, tautomer, or solvate thereof

In addition, ATA may be in form of ATA analogs, derivatives, or chemotypes thereof. As used herein, the term “aurintricarboxylic acid analogs, derivatives, or chemotypes thereof” or “ATA analogs, derivatives, or chemotypes thereof” includes chemically modified or structurally variants thereof that act as UDGis. That is, compounds having the same general structure as ATA, its oligo- and polymers and its rearrangement products and further having the same biological activity as ATA and pharmaceutically acceptable salts, tautomers, or solvates thereof, wherein “biological activity” refers to the binding of the compound to UDG such that the UDG is maintained in a destabilized, open precatalytic glycosylase conformation that prevents active site closing for functional DNA binding and nuclease flipping needed to excise damaged bases binding in DNA. Suitable tests for detecting the biological activity, namely, the inhibitory activity on adherence is described in the Examples.

In some embodiments, ATA analogs, derivatives, and chemotypes thereof of ATA include substituted nonsymmetric ATA compounds having formula (II):

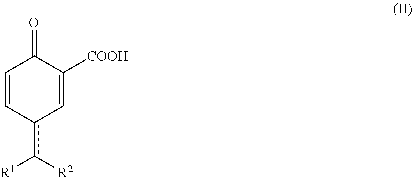

or a pharmaceutically acceptable salt, tautomer, or solvate thereof;
wherein:
R¹ and R² are each independently H, halogen, hydroxyl, an optionally substituted alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety;
---- is an optional bond; and
at least one of R¹ or R² is an optionally substituted salicylic acid, salicylate, or salicylic ester.

In other embodiments, only one of R¹ or R² is an optionally substituted salicylic acid, salicylate, or salicylic ester.

In some embodiments, ATA analogs, derivatives, and chemotypes thereof include substituted nonsymmetric ATA compounds having formula (III):

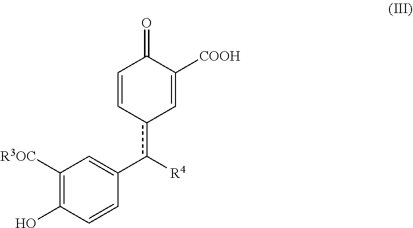

or a pharmaceutically acceptable salt, tautomer, or solvate thereof;
wherein:
--- is an optional bond;
R³ is OH, alkyl, haloalkyl, thiol, thiol alkyl, or alkylene-OH each of which is optionally substituted; and
R⁴ is H, halogen, hydroxyl, an optionally substituted alkyl, alkenyl, alkynyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, alkylamino, dialkylamino, arylamino, diaryl amino, alkylaryl amino, acylamino, alkylcarbonylamino, arylcarbonylamino, carbamoyl, ureido, amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, aryl, heterocyclyl, heteroaryl, alkylaryl, aromatic moiety, or heteroaromatic moiety.

In some embodiments, ATA analogs, derivatives, and chemotypes thereof include substituted nonsymmetric ATA compounds having formula (IV):

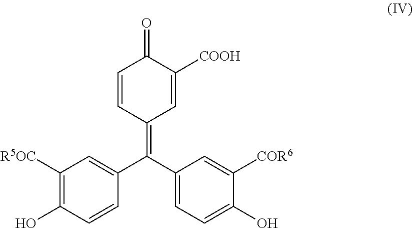

or a pharmaceutically acceptable salt, tautomer, or solvate thereof;
wherein:
--- is an optional bond; and
R⁵ and R⁶ are each independently OH, alkyl, haloalkyl, thiol, thiol alkyl, or alkylene-OH each of which is optionally substituted.

In some embodiments, R⁵ and R⁶ are not the same.

In other embodiments, ATA analogs, derivatives, and chemotypes thereof can include nonsymmetric ATA compounds including substituted ATA compounds wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

In particular embodiments, ATA analogs, derivatives, and chemotypes thereof include nonsymmetric ATA compounds wherein the carboxylic group is esterified or the hydroxyl groups are substituted with substituents, thiol groups, alkyl groups, or acyl groups, such as described for example, in U.S. Pat. Nos. 4,007,270 and 5,434,185, as well as U.S. Patent Application Publication No. 2014/0329780 all of which are incorporated by reference in their entirety.

In some embodiments, the agent or UDGi can be administered to a subject in combination with an antimetabolite agent and optionally a BER inhibitor, such as an AP endonuclease inhibitor to mitigate side-effect burdens on the patient being treated.

The antimetabolite agent can include agents, compounds, or small molecules that induce or promote incorporation of a UDG substrate, such as uracil, into DNA of cancer cells of the subject. Antimetabolite agents include, but are not limited to acanthifolic acid, aminothiadiazole, anastrozole, bicalutamide, brequinar sodium, capecitabine, carmofur, Ciba-Geigy CGP-30694, cladribine, cyclopentyl cytosine, cytarabine phosphate stearate, cytarabine conjugates, cytarabine ocfosfate, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, finasteride, floxuridine, fludarabine, fludarabine phosphate, N-(2′-furanidyl)-5-fluorouracil, Daiichi Seiyaku FO-152, fluorouracil (5-FU), 5-FU-fibrinogen, isopropyl pyrrolizine, Lilly LY-188011, Lilly LY-264618, methobenzaprim, methotrexate, Wellcome MZPES, nafarelin, norspermidine, nolvadex, NCI NSC-127716, NCI NSC-264880, NCI NSC-39661, NCI NSC-612567, Warner-Lambert PALA, pemetrexed pentostatin, piritrexim, plicamycin, Asahi Chemical PL-AC, raltitrexed stearate; Takeda TAC-788, thioguanine, tiazofurin, Erbamont TIF, trimetrexate, tyrosine kinase inhibitors, tyrosine protein kinase inhibitors, Taiho UFT, toremifene, and uricytin, all of which are disclosed in U.S. Pat. No. 6,916,800, which is herein incorporated by reference in its entirety.

In some embodiments, the antimetabolite agent can be a thymidylate synthase (TS) inhibitor that when administered to a cancer cell of a subject promotes incorporation of a UDG substrate into the DNA of the cell. One example of a thymidylate synthase that is an antimetabolite and induces or promotes incorporation of a UDG substrate, such as uracil, into DNA of cancer cells is 5-fluorouracil (5-FU). 5-Fluorouracil has been used clinically in the treatment of malignant tumors, including, for example, carcinomas, sarcomas, skin cancer, cancer of the digestive organs, and breast cancer. 5-Fluorouracil, however, can cause serious adverse reactions, such as nausea, alopecia, diarrhea, stomatitis, leukocytic thrombocytopenia, anorexia, pigmentation, and edema. Derivatives of 5-fluorouracil with anti-cancer activity have been described in U.S. Pat. No. 4,336,381. Further 5-FU derivatives have been described in the following patents listed in JP 50-50383, JP 50-50384, JP 50-64281, JP 51-146482, and JP 53-84981 hereby individually incorporated by reference herein.

In other embodiments, the antimetabolite agent can be an antifolate agent that when administered to a cancer cell of a subject promotes incorporation of a UDG substrate into the DNA of the cell. An example of an antifolate agent is pemetrexed. Pemetrexed inhibits several key folate-dependent enzymes in the thymidine and purine biosynthetic pathways, including thymidylate synthase, dihydrofolate reductase, and glycinamide ribonucleotide formyltransferase. As an analogue of methylenetetrahydrofolate, pemetrexed directly blocks dTMP production by depleting tetrahydrofolate pools required for TS. In comparison to other anti-metabolites, pemetrexed is the most potent inducer of uracil incorporation into DNA.

In still other embodiments, the antimetabolite agent can be a nucleoside analogue that when administered to a cancer cell of a subject promotes incorporation of a UDG substrate into the DNA of the cell. In some examples, the nucleoside analogue can be 2-Fluoroadenosine-5′-phosphate or fludarabine (F-ara-A). Fludarabine is one of the most active agents in the treatment of chronic lymphocytic leukemia. The compound acts by inhibiting DNA synthesis. Treatment of cells with fludarabine is associated with the accumulation of cells at the G1/S phase boundary and in S phase; thus, it is a cell cycle S phase-specific drug. Incorporation of the active metabolite, F-araATP, retards DNA chain elongation. Fludarabine is also a potent inhibitor of ribonucleotide reductase, the key enzyme responsible for the formation of dATP.

Alternatively, the UDGi and antimetabolite agent can be administered in combination with an AP endonuclease to promote or enhance the cytotoxicity of the UDGi and antimetabolite agent. As discussed above, administration to a cancer cell of an antimetabolite in combination with a UDG inhibitor and optionally an AP endonuclease inhibitor can enhance antimetabolite induced cell death. This enhances the cytotoxicity of the antimetabolite agents and UDGi by further inhibition of the BER pathway and allows treatment of cancers that express high levels of UDG that were previously found to be resistant to treatment with antimetabolite agents.

The AP endonuclease inhibitor that potentiates the cytotoxicity of the UDGi and antimetabolite agent can be a small molecule compound with a primary amine group that forms a covalent linkage with and/or binds to an aldehyde group of an AP site induced by the antimetabolite agent. In single-nucleotide BER, the deoxyribose phosphate (dRP) in the abasic site is removed by the lyase activity of DNA pol β. Binding of the AP endonuclease inhibitor to an aldehyde group can structurally alter the AP site so that AP endonuclease does not recognize the modified AP site and/or prevent AP endonuclease-mediated cleavage of phosphodiester bonds, thus blocking single nucleotide BER.

In some embodiments, the reaction of the AP endonuclease inhibitor with the aldehyde group in the cancer cells can be faster than AP endonuclease to inhibit repair of DNA. Advantageously, administration of the AP endonuclease inhibitor in combination with the antimetabolite agent and/or UDG inhibitor to tumor cells can bypass other resistance factors, such as MMR defects and high MGMT activity in the tumor cells.

In some embodiments, the AP endonuclease inhibitor can be an aminooxy small molecule that can react with an AP site faster than AP endonuclease. One example of an aminooxy compound that that can react with an AP site faster than AP endonuclease is methoxyamine (MX) or salts thereof. Methoxyamine when administered in combination with a UDGi, such as ATA, and an antimetabolite agent, such as pemetrexed, to a subject with cancer can potentiate the anticancer effect of the UDGi and antimetabolite agent without additive systemic toxicity.

Other examples of small molecules primary amine compounds that can bind to AP sites and prevent APE-mediated cleavage of phosphodiester bonds can be identified using a high-throughput screening assay described in U.S. Pat. Nos. 8,367,332, 8,324,282, 6,635,677, and 6,465,448, all of which are incorporated by reference in their entirety.

In some embodiments, the UDGi can be administered to an individual in combination with the antimetabolite and optional AP endonuclease inhibitor. For example, the UDGi, antimetabolite agent, and optional AP endonuclease inhibitor can be administered to an individual together in a parenteral formulation. Alternatively, the UDGi, antimetabolite agent, and optional AP endonuclease inhibitor can be administered to an individual together in an oral formulation, such as a solid dosage formulation.

In some embodiments, the UDGi, antimetabolite agent, and optional AP endonuclease inhibitor can be administered to an individual sequentially, where the individual is first given the antimetabolite agent and then given the UDGi and optional AP endonuclease inhibitor. For example, the individual can be given the antimetabolite agent in a parenteral formulation, such as an intravenous formulation, or an oral formulation, such as a solid dosage formulation, and then given the UDGi and optional AP endonuclease inhibitor in a parenteral formulation, such as an intravenous formulation, or an oral formulation, such as a solid dosage formulation.

Alternatively, in some embodiments, the UDGi, antimetabolite agent, and optional AP endonuclease inhibitor can be administered to an individual sequentially, where the individual is first given the UDGi and/or optional AP endonuclease inhibitor and then given the antimetabolite agent. For example, the individual can be given the UDGi and optional AP endonuclease inhibitor in a parenteral formulation, such as an intravenous formulation, or an oral formulation, such as a solid dosage formulation and then given the antimetabolite agent in a parenteral formulation, such as an intravenous formulation, or an oral formulation, such as a solid dosage formulation.

In some embodiments, the UDGi, antimetabolite agent, and optional AP endonuclease inhibitor can create an anticancer effect greater than that of the separate anticancer effects of the individual agents. For example, the combined anticancer effect of the UDGi, antimetabolite agent, and optional AP endonuclease inhibitor can be greater than the added anticancer effect of the UDGi, antimetabolite agent, and optional AP endonuclease inhibitor when used individually.

In certain embodiments, an antimetabolite agent, such as pemetrexed, that induces incorporation of uracil into DNA of the cancer can be administered in combination with a UDGi, such as ATA, and an AP endonuclease inhibitor, such as methoxyamine, after it is determined that cancer of subject has an increased level of UDG expression compared to a control level.

In some embodiments, the antimetabolite agent can be administered in a dose of from about 10 mg/m² to about 5,000 mg/m² body surface area. For example, the dose can be from about 20 mg/m² to about 200 mg/m² body surface area; the dose can be from about 150 mg/m² to about 500 mg/m² body surface area; the dose can be from about 400 mg/m² to about 1000 mg/m² body surface area; the dose can be from about 900 mg/m² to about 5,000 mg/m² body surface area; the dose can be from about 200 mg/m² to about 1,000 mg/m² body surface area; or the dose can be from about 500 mg/m² to about 600 mg/m² body surface area. In some embodiments, the antimetabolite agent can be pemetrexed and pharmaceutically acceptable salts thereof.

In some embodiments, the UDGi and optional AP endonuclease inhibitor is administered in an amount efficient to enhance or increase the effect of an antimetabolite agent.

In other embodiments, the UDGi, antimetabolite agent, and optional AP endonuclease inhibitor can be administered to subject in combination with at least one other BER inhibitor. The at least one other BER inhibitor can include, for example, a PARP inhibitor. Examples of PARP inhibitors are 4-amino-1,8-naphthalimide (ANI), PD128763, 3-AB, 6-AN, and 8-hydroxy-2-methyl-quinazolin-4-[³H]one (NU-1025).

Other examples of BER inhibitors that can be administered to the subject in combination with the UDGi, antimetabolite agent, and optional AP endonuclease inhibitor include DNA polymerase inhibitors (e.g., DNA polymerase β, γ or ε), such as prunasin, aphidicolin, 2′,3′-dideoxycytidine triphosphate (ddCTP), 2′,3′-dideoxythymidine triphosphate (ddTTP), 2′,3′-dideoxyadenosine triphosphate (ddATP), 2′,3′-dideoxyguanosine triphosphate (ddGTP), 1-beta-D-arabinofuranosylcytosine (Ara-C), caffeine, arabinocytidine, and bleomycin.

Still other examples of BER inhibitors include DNA ligase inhibitors (e.g., DNA ligase I, II, or III), such as ursolic and oleanolic acids, aleuritolic acid, protolichesterinic acid, swertifrancheside, fulvoplumierin, fagaronine chloride, and bleomycin. XRCC1 is the protein partner of DNA ligase III, and inhibitors of XRCC1, such as 3-AB, are useful as BER inhibitors as well.

Further examples of BER inhibitors include topoisomerase II inhibitors. Topoisomerase inhibitors induce DNA cleavage and other chromosomal aberrations, including sister chromatid exchanges. Compounds useful as BER inhibitors also include topoisomerase II inhibitors, such as etoposide (VP-16, VP-16-123), meso-4,4′-(2,3-butanediyl)-bis-(2,6-piperazinedione) (ICRF-193, a bisdioxopiperazine), doxorubicin (DOX), L amsacrine (4′,9-acridinylaminomethanesulfon-m-anisidide; mAMSA), pazelliptine, nalidixic acid, oxolinic acid, novobiocin, coumermycin A1, fostriecin, teniposide, mitoxantrone, daunorubicin, N-[2-dimethylamino)ethyl]acridine-4-carboxamide (DACA), merbarone, quinacrine, ellipticines, epipodophyllotoxins, ethidium bromide, epirubicin, pirarubicin, 3′-deamino-3′-morpholino-13-deoxo-10-hydroxy caminomycin; 2″,3″-bis pentafluorophenoxyacetyl-4′,6′-ethylidene-beta-D glucoside of 4′-phosphate-4′-dimethylepipodophyollotoxin 2N-methyl glucamine salt (F11782; a fluorinated lipophilic epipodophylloid), adriamycin, actinomycin D, anthracyclines (such as 9-aminoanthracycline), and pyrazoloacridine (PZA). Topoisomerase I inhibitors, such as camptothecin and topotecan can also be used as BER inhibitors.

In some embodiments, other enzyme inhibitors, whether known in the art or hereafter identified, as well as inhibitors of other elements of the BER pathway, such as DNA alkyltransferase, may be employed in compositions and methods without departing from the scope and spirit of the present embodiments.

In still other embodiments, the UDGi, antimetabolite agent, and optional AP endonuclease inhibitor can be administered to subject in combination with at least one other anticancer agent that induces formation of AP sites. Anticancer agents that induce the formation of AP sites include intercalating agents, such as bleomycin, adriamycin, quinacrine, echinomycin (a quinoxaline antibiotic), and anthrapyrazoles.

Radiation, such as gamma radiation, UVA, and UVB, can be used to generate AP sites. Ultraviolet light is absorbed in DNA with the formation of UV-specific di-pyrimidine photoproducts. Exposure to gamma irradiation, UVA, and UVB can induce damaged pyrimidine photodimers. Anticancer agents that induce the formation of AP sites can also include DNA oxidizing agents, such as hydrogen peroxide.

Anticancer agents that induce the formation of AP sites can further include alkylating agents, such as temozolomide (TMZ), 1,3-bis(2-chloroethyl)-1-nitrosourea (BCNU), MeOSO₂(CH₂)₂-lexitropsin (Me-Lex), cis-diamminedichloroplatinum II (cisplat; cis-DDP), mitomycin bioreductive alkylating agents, quinones, streptozotocin, cyclophosphamide, nitrogen mustard family members such as chloroambucil, pentostatin (and related purine analogs), fludarabine, bendamustine hydrochloride, chloroethylating nitrosoureas (e.g., lomustine, fotemustine, cystemustine), dacarbazine (DTIC), and procarbazine. In certain embodiments, the alkylating agent is a nitrosoruea, such as a mustine.

Alkylating agents can function by adding methyl groups to DNA, cross-linking macromolecules essential for cell division, and linking guanine bases in DNA through their N⁷ atoms. Both inter- and intra-strand cross-links can be mediated by alkylating agents. Inter-strand cross-links prevent the separation of the DNA strands necessary for cell division, and by being more difficult to repair, constitute the more lethal lesion.

In certain embodiments, the anticancer agent is selected from radiosensitizers such as 5-iodo-T-deoxyuridine (IUdR), 6-thioguanine, hypoxanthine, uracil, ecteinascidin-743, and camptothecin and analogs thereof.

It will be appreciated that compositions or formulations provided herein may be in any form, which allows for the composition to be administered to a patient. For example, the composition may be in the form of a solid, liquid or gas (e.g., aerosol). Other routes of administration include, without limitation, oral, topical, parenteral (e.g., sublingually or buccally), sublingual, rectal, vaginal, and intranasal. The term parenteral as used herein includes subcutaneous injections, intravenous, intramuscular, intrasternal, intracavemous, intrathecal, intrameatal, intraurethral injection or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units.

Pharmaceutical compositions can include physiologically acceptable surface active agents, carriers, diluents, excipients, smoothing agents, suspension agents, film forming substances, and coating assistants, or a combination thereof; and a compound disclosed herein. Acceptable carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives, stabilizers, dyes, sweeteners, fragrances, flavoring agents, and the like may be provided in the pharmaceutical composition. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium methasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example, dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Routes of administration may, for example, include oral, rectal, transmucosal, topical, or intestinal administration; parenteral delivery, including intramuscular, subcutaneous, intravenous, intramedullary injections, as well as intrathecal, direct intraventricular, intraperitoneal, intranasal, or intraocular injections. The compounds can also be administered in sustained or controlled release dosage forms, including depot injections, osmotic pumps, pills, transdermal (including electrotransport) patches, and the like, for prolonged and/or timed, pulsed administration at a predetermined rate.

The pharmaceutical compositions of the present invention may be manufactured in a manner that is itself known, e.g., by means of conventional mixing, dissolving, granulating, dragee-making, levigating, emulsifying, encapsulating, entrapping or tableting processes.

Pharmaceutical compositions for use in accordance with the present invention thus may be formulated in conventional manner using one or more physiologically acceptable carriers comprising excipients and auxiliaries, which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations (for example, liposomes), may be utilized.

For transmucosal administration, penetrants appropriate to the barrier to be permeated may be used in the formulation.

Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Additional therapeutic or diagnostic agents may be incorporated into the pharmaceutical compositions. Alternatively or additionally, pharmaceutical compositions may be combined with other compositions that contain other therapeutic or diagnostic agents.

The compounds or pharmaceutical compositions may be administered to the patient by any suitable means. Non-limiting examples of methods of administration include, among others, (a) administration though oral pathways, which administration includes administration in capsule, tablet, granule, spray, syrup, or other such forms; (b) administration through non-oral pathways such as rectal, vaginal, intraurethral, intraocular, intranasal, or intraauricular, which administration includes administration as an aqueous suspension, an oily preparation or the like or as a drip, spray, suppository, salve, ointment or the like; (c) administration via injection, subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; as well as (e) administration topically; as deemed appropriate by those of skill in the art for bringing the compound of the invention into contact with living tissue.

Pharmaceutical compositions suitable for administration include compositions where the active ingredients are contained in an amount effective to achieve its intended purpose. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be between about 10 microgram/kg and 100 mg/kg body weight, preferably between about 100 microgram/kg and 10 mg/kg body weight. Alternatively, dosages may be based and calculated upon the surface area of the patient, as understood by those of skill in the art.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, which is hereby incorporated herein by reference in its entirety, with particular reference to Ch. 1, p. 1). Typically, the dose range of the composition administered to the patient can be from about 0.5 to 1000 mg/kg of the patient's body weight. The dosage may be a single one or a series of two or more given in the course of one or more days, as is needed by the patient. In instances where human dosages for compounds have been established for at least some condition, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency will also vary according to the age, body weight, and response of the individual patient. A program comparable to that discussed above may be used in veterinary medicine.

The following examples is included to demonstrate preferred embodiments of the invention. It should be appreciated by those of skill in the art that the techniques disclosed in the example, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE

This example describes an optimized high-throughput (HTS) screening platform used to identify and characterize a preclinical UDG inhibitor (UDGi) for cancer biology. Structural and computational analyses reveal that the identified inhibitor binds to the open UDG active site conformation. As opposed to canonical inhibitors based upon transition state mimicry, these results uncover tactical advantages for BER enzymes, such as UDG, to instead block the conformational closing needed for damaged DNA substrate recognition and catalysis. By analogy, the identified UDGi acts as a “door stopper” keeping the active site open such that the closed conformation needed for productive DNA binding plus “lock and key” substrate recognition and catalysis by UDG is precluded.

### ATA Structural and Chemical Attributes

The cell-based assays described herein validate the inhibition of human UDG by ATA by defining its mechanism of inhibition and functionality. We reasoned that as ATA provides a distinct chemotype from uracil its ability to bind nucleic acid enzymes might prove advantageous. Our identification of the structural mechanism whereby ATA holds UDG in its open destabilized configuration provides an initial chemotype for a “door stopper” strategy to guide design of more specific UDG inhibitors. In contrast, binding uracil promotes the closed DNA-binding conformation of UDG. So we reason that uracil-like compounds may have larger challenges to avoid being displaced by the larger DNA interface with the entire active site channel compared to compounds guided by our ATA complex with open UDG.

### Methodology

### High-Throughput Small-Molecule Screening (HTS) Biochemical Assay

The collection of biologically active molecules was compiled from the LOPAC library (Sigma Aldrich) and Bioactive Compound Library (Selleck Chemicals). A total of 3115 mechanistically annotated compounds were used for screening. All stock solutions were prepared in dimethyl sulfoxide (DMSO) at 3 mM. Upon hits identification, all compounds were purchased from an original vendor and retested from 10 mM stock solutions in DMSO. For the screening, 384-well assay plates were prepared with final test concentrations of 12.5 μM using a Janus liquid handling platform (Perkin Elmer) equipped with a 50 nL pin transfer tool (V&P Scientific). For concentration-response studies at eight concentrations in half-log dilutions, final test plates were prepared from 10 mM DMSO stock solutions using Janus liquid handling platform (Perkin Elmer) equipped with a standard 96 tip head. A final DMSO concentration of 0.125% was not exceeded in the screening assay and hit validation. The negative controls contained the same percentage of the vehicle. Small molecule solutions were transferred to a 384 well microtiter plate (Corning 3573) where each well contained 20 μL of reaction buffer (2×: 40 mM Tris HCl, 100 mM KCl, 0.4 mM MgCl₂, 2 mM DTT, 0.1% Tween), 50 nL of 10 mM compound stock in 100% DMSO, 10 μL of human UDG protein (40 nM stock, Creative BioMart company recombinant human mitochondrial UDG, GST tagged, species human, source E. coli), 10 μL of double-stranded 14-mer DNA hairpin that contains nine U:A base pairs (100 nM stock, 5′FAM-GCA CUU AAG AAU UGC AAU UCU UAA GUG C-3′Dabsyl, Eurofins Genomics). After 40 min of incubation at room temperature (˜23° C.), fluorescence measurements were done using an Enspire plate reader (Perkin Elmer) at an excitation wavelength of 485 nm and emission wavelength of 520 nm. Compound performance from per plate was calculated and normalized accounting for background fluorescence by the following equation:

% Activity=[RFU(compound well)−RFU-UDG protein AVG(DNA alone)]/[RFU+UDG AVG(DMSO controls)−RFU-UDG protein AVG(DNA alone)]×100. Signal over background was determined for each plate, and Z prime was calculated based on the following equation:Z prime=1−[(3SD of sample+3SD of control)/(mean of the sample−mean of control)](Zhang et al., 1999).

### Multiple Uracil-Containing DNA Melting

### DNA Hairpins:

| (SEQ ID NO: 1) | |
|---|---|
| | 5′FAM- GCA CTT AAG AAT TGC AAT TCT TAA GTG C- |
| | 3′Dabcyl |
| | (SEQ ID NO: 2) |
| | 5′FAM- GCA CUU AAG AAU UGC AAU UCU UAA GUG C- |
| | 3′Dabcyl |

DNA was diluted to 1000 nM stock (in water). DNA hairpin was heated to 50° C. for 20 min and allowed to cool for at least 60 min to room temperature. Each reaction (total 40 μL) included: 15.5 μL of ddH₂0, 4 μL of buffer (2 mM TRIS-HCl, 5 mM KCl, 0.02 MgCl₂, 0.1 mM DTT, 0.005% Tween), 0.5 μL of 100% DMSO or compound. Pre-incubation took place for 20 min at room temperature. Then, 20 μL of DNA hairpin (1000 nM stock) was added to initiate the reaction. A transparent sealant was applied to seal the plate and incubation occurred for 45 min at room temperature. Each plate was spun down gently for 20s and was loaded onto an RT-PCR machine (BioRad CFX96, FAM filter). FAM fluorescence intensity was monitored every 1.6° C./2 min from 25° C.-97° C. RFU and negative derivative RFU raw data and plots from the machine were obtained and analyzed. Melting temperature (Tm) value was taken directly from the midpoint on the−dRFU/dt vs. temperature graph.

### UDG-ATA Co-Crystallization, Data Collection, and Structure Refinement

UDG was expressed, purified following a published protocol. ATA compound was dissolved in water. We grew crystals using the hanging drop method under co-crystallization of the UDG protein construct and the ATA at a concentration in solution of 4 mM. Crystallization condition was 2 mM TRIS pH 8.5, 20 mM MgCl₂, 21% PEG8000 at 291 K. Diffraction data were collected, processed, and refined as described in Supplemental Information. X-ray data for crystallography were collected at SSRL BEAMLINE BL14-1 refined structures have useful statistics and geometry (Table 1). The refined X-ray crystal structure with diffraction data for UDG and bound ATA is deposited in the Protein Data Bank, PDB ID is 6VBA.

TABLE 1
X-ray diffraction data collection and refinement statistics

| | UDG-ATA |
|---|---|
| Data collection | |
| Space group | P 21 21 2 |
| Cell dimensions [symbol] | |
| a, b, c (Å) | 73.65, 54.78, 59.99 |
| [symbol] (°) | 90, 90, 90 |
| Resolution (Å) | 35.45-1.8 |
| R_{ayo:} or R_{merge} | 12.5 |
| I/ [symbol] I | 15 |
| Completeness (%) | 91.91 |
| Redundancy | 11 2 |
| Refinement | |
| Resolution (Å) | 1.8 |
| No. reflections | 21264 |
| R_{work}/ R [symbol] | 17.92/ 23.36 |
| No. atoms | |
| Protein | 1808 |
| Ligand/ion | 62 |
| Water | 259 |
| B-factors | |
| Protein | 23.07 |
| Ligand/ion | 89.20 |
| Water | 31.76 |
| R.m s. deviations | |
| Bond lengths (Å) | 0.0036 |
| Bond angles (°) | 0.8775 |

[symbol]

indicates data missing or illegible when filed

### Molecular Docking

We selected a series of structures of UDG from APO to complexes to have a broad spectrum of results to analyze. Specifically, we used: PDB 1AKZ which is the APO structure solved at 1.57 Å, PDB 1UGH which is the UDG-Ugi complex solved at 1.9 Å(Mol et al., 1995a), PDB 1SSP which is a product complex and uracil solved at 1.9 Å(Parikh et al., 1998), and PDB 1EMH which is the complex of UDG-dsDNA-pseudouracil solved at 1.8 Å. We superposed all structures to UDG Chain of 1SSP to compare the population of docking results from different results aligned to specific cofactors. Each structure was prepared for docking by eliminating water and cofactors; specifically, for 1UGH, we eliminated the Ugi chain; for the 1SSP, we eliminated the DNA substrates chains and uracil and for 1EMH the ds-DNA substrate. The UDG coordinates for each structure were prepared and minimized using Protein Preparation wizard. We focused molecular docking to the uracil binding active site region, and we selected the residue GLN144 to prepare the docking grid for all structures. We docked ligands with length ≤20 Å with cubic box dimensions 15 Å. Ligands used for this study were Uracil, Salicylic acid, and Aurintricarboxylic acid (ATA). We downloaded the files which contain the atoms and topology of the ligands from PubChem (https://pubchem.ncbi.nlm.nih.gov); we then prepared the ligands for docking experiments using LigPrep. We ran a single docking experiment for each ligand to each of the four prepared UDG structures. For the docking experiments, we used Glide Extra Precision (XP). Each of ligand conformations generated by LigPrep has been automatically docked using flexible docking and ranked based on docking score. The best docking results were selected and compared in different superposed PyMOL sessions (PyMOL, The PyMOL Molecular Graphics System, Version 2.0 Schrödinger, LLC.) to generate figures and 2D ligand-receptor interaction diagram. All computational suite is provided by SB Grid Consortium.

### Microscale Thermophoresis (MST)

MST was performed to measure the binding affinity between ATA and UDG using the Monolith NT.115 from Nanotemper Technologies. Bacterially expressed and purified full-length human UDG was fluorescently labeled with Atto488 NHS ester according to the manufacturer's protocol. Briefly, 0.5 mg of protein in 20 mM HEPES, 150 mM NaCl pH8.0 (final volume 3000 was mixed with 10 μl Atto488 NHS ester (2 mg/ml in DMSO) and incubated at room temperature for an hour. Equilibrated PD10 desalting column was used to separate labeled proteins from the free dye. Labeling efficiency was determined to be 1:1 (protein to dye) by measuring the absorbance at 280 and 488 nm and the concentration of protein relative to the dye. A solution of ATA was serially diluted from about 25 μM to 12 nM in the presence of fixed concentration of 100 nM Atto488-labeled UDG in 0.01 M HEPES pH 7.4, 0.15 M NaCl, 0.005% v/v Surfactant P20. The samples were loaded into silica premium capillaries (Polymicro Technologies) after incubation at room temperature for 15 min measurements were performed at 20° C., by using 20% LED power and 40% IR-laser power. Measurements were also carried out on 40 and 60% IR-Laser power for comparison. The raw data were exported to GraphPad Prism8 software, KD value was obtained by data fitting with a one-site specific binding model.

### Differential Scanning Fluorimetry Assay

Differential scanning fluorimetry (DSF) measurements were performed on a Prometheus NT.48 instrument (NanoTemper Technologies, Munich, Germany). Full-length UDG at 14 μM in 137 mM NaCl, 2.7 mM KCl, 10 mM Na₂HPO₄, and 1.8 mM KH₂PO₄ pH 7.4 was combined with titrating concentration of ATA mixed, incubated at room temperature for 10 min. The capillaries were filled with 10 μL sample and placed on the sample holder. A temperature gradient of 1° C.·min⁻¹ from 25 to 90° C. was applied and the intrinsic protein fluorescence at 330 and 350 nm was recorded. Additional aggregation onset temperatures are detected via back-reflection light scattering. ATA binding induced neither a red-shift nor a blue-shift upon protein unfolding caused by an overall decrease in fluorescence intensity in both 330 nm and 350 nm channels and producing a flat ratio curve. Therefore, analysis of single wavelength was used to determine the melting temperature.

### Validating ATA Inhibitory Effect on UDG DNA Cutting Activity

Biochemical DNA Cleavage Assay with a Single oxoG:C Basepair and a Single U:A Basepair Sequences: DNA Duplexes

To a 96 well microtiter plate (polypropylene) these reaction components were added: 10 μL of UDG buffer (stock 100 mM Tris-HCl, 250 mM KCl, 1 mM MgCl₂, 5 mM DTT, 0.25% Tween), 32 μL of ddH₂0, 2 μL of DMSO/ATA stock (312 μM, 104 μM, 35 μM or 11.6 μM), and 4 μL of UDG (6.25 nM stock, XTAL Biostructures company recombinant human mitochondrial UDG, 6xhis tagged, species was human, source was Sf9 insect cells). Following pre-incubation occurred for 20 min at 37° C., 2 μL of U:A DNA (500 nM stock) was added to initiate the reaction, total reaction volume of 50 μL/well. Each plate was incubated for 120 min at 37° C. Then, each 96 well plate was sealed and heated at a constant temperature of 95° C. for 55 min using the CFX96 real time system thermocycler. After the heating process, 5 μL of a bromophenol blue denaturing loading dye (20milligrams of bromophenol blue, 9.7 ml of formamide plus 300 μL of 10M NaOH) was added to each reaction well. DNA products, from each reaction, were resolved on 20% polyacrylamide nucleic acid denaturing gel (UreaGel 29:1 System EC-829, 7.5M urea+1×TBE, National diagnostics). TBE buffer was prepared by dissolving 54 grams of tris base, 28 grams of boric acid, and 4.5 grams of EDTA in 1.0 liter of distilled H₂O. Gels were processed at 200V for 60 min in the dark. Gels were imaged based on the fluorescent tags using Typhoon Trio+Variable Mode Imager (Amersham Biosciences) with excitation and emission wavelengths set to 532 nm and 585 nm respectively, photomultiplier tube voltage (PMT) was set to 400V, and pixel size resolution set to 100 μm. Gel data were analyzed using ImageJ software. DNA bands were corrected using a background subtraction step. Percent cleavage was calculated by using fluorescence intensity of the cleaved 23-mer DNA strand divided by the sum of the fluorescence bands (23mer+40mer DNA strands)*100. Relative percent cleavage was generated by using the untreated DMSO condition divided by each treated drug dose*100. Reactions with OGG1 (protein stock from NEBiolabs: humanOGG1) were processed similarly as described above with a different reaction buffer recipe (100 mM TrisHCl, 250 mM KCl, 2 mM MgCl₂, 5 mM DTT, 0.25%, 0.5 μg/ul of BSA, with OGG1 protein stock of 45 nM, with 1 μL of oxoG:C DNA (500 nM stock), with a total reaction volume of 50 μL/well and an incubation time of 180 min at 37° C. The IC50 value was generated using Prism software version 8.4.3, Y=bottom+X*(Top-Bottom/(IC₅₀+X), graphpad.com, non-linear fit, [inhibitor] vs. response (three parameters, R2=0.98).

Cell-Free Protein Extracts from Human DLD1 Cancer Cell Lines

Total protein lysates were extracted following the manufacturer's recommended protocol (Millipore CytoBuster protein extraction reagent #71009-3) from human colon DLD1 cancer cell lines: cell lines expressing UDG (wild type and shScramble), cell line with an sh-knockdown UNG (shKD-UNG), and a cell line with a CRISPR-knockout UDG (KO-UNG). Each 96 well plate contained 10 μL of reaction buffer (final concentration of 20 mM Tris HCl, 50 mM KCl, 0.2 mM MgCl₂, 1 mM DTT, 0.05% Tween), distilled H₂O, either 2 μL of DMSO or ATA and 20 μg of total whole cell lysate. The reaction was pre-incubated for 20 min at 37° C. before a fluorescently-labeled TAMRA DNA duplex oligo containing a single U:A bp (final 30 nM) was added to each reaction well with a total reaction volume of 50 μL/well. Plate was further incubated for 20 min at 37° C. 5 μL of a denaturing loading dye (20 mg of bromophenol blue, 9.7 ml of formamide plus 300 μL of 10M NaOH) was added to stop each reaction. DNA products from each reaction were resolved on a 20% denaturing polyacrylamide nucleic acid urea gel (5.3 grams of urea was dissolved in 2.3 mL of 5×TBE, 5 mL of 40% bis-acrylamide 29:1 solution). 5×TBE buffer was prepared by dissolving 54 grams of tris base, 28 grams of boric acid, and 4.5 grams of EDTA in a liter of distilled H₂O. Gels were processed at 200V for 60 mins at room temperature, in the dark. Gels were imaged based on the fluorescent tags using Typhoon Trio+Variable Mode Imager (Amersham Biosciences) with excitation and emission wavelengths set to 532 nm and 585 nm respectively, PMT set to 400, and pixel size resolution set to 100 m. Gel data were analyzed using ImageJ software. DNA bands were corrected using a constant background band intensity subtraction step. Then, percent cleavage was calculated by using fluorescence intensity of the cleaved 23-mer DNA strand divided by the sum of the fluorescence bands (23mer+40mer DNA strands)*100 for untreated and treated conditions. Relative percent cleavage was generated by using each treated drug dose divided by the untreated DMSO condition*100. The IC50 value was estimated using Prism software version 8.4.3, non-linear fit, Y=100*(X\hat{ }HillSlope)/(IC50\hat{ }HillSlope+(X\hat{ }HillSlope)), graphpad.com, [inhibitor] vs. normalized response (variable slope, R2=0.97) averaging points from four different gels.

### Measurement of Uracil Removal in Reporter Plasmids in Cells

Removal of uracil in cells was measured using a reporter plasmid containing a dU:dG base pair (referred to as BFP_U), as described previously. In brief, dU is substituted into the transcribed strand at position 191 of the blue fluorescent protein (BFP) gene. When RNA polymerase II incorporates dA opposite dU, transcripts encoding wild type BFP are produced. Upon repair, dU is replaced by dC, leading to production of transcripts encoding a non-fluorescent protein. Hence, fluorescent signal is directly proportional to the dU presence in the reporter plasmid. The reporter plasmid (100 ng per transfection) was mixed with a transfection control plasmid (pmax GFP, 100 ng per transfection) along with a carrier plasmid (800 ng per transfection) that does not produce fluorescent protein to constitute the “uracil repair” cocktail. A corresponding cocktail of damage-free plasmids containing pmax BFP (100 ng per transfection), pmax GFP (100 ng per transfection), and carrier plasmid (800 ng per transfection) constituted the “undamaged” cocktail. After normalizing to expression of the pmax,GFP transfection control, expression of BFP_U in the damaged cocktail was normalized to similarly normalized expression of pmax_BFP in the undamaged cocktail. The reciprocal of BFP_U reporter expression was calculated for each experimental condition. Finally, the reciprocal of BFP_U reporter expression for each inhibitor concentration was normalized to reciprocal of BFP_U reporter expression for cells treated with vehicle alone (DMSO). Reporter plasmids for measuring removal of hypoxanthine, 8-oxoG opposite dC, and adenine opposite 8-oxoG were as reported previously. The reporter plasmid for measuring removal of thymidine opposite dG operates under the same principle as BFP_U and the other glycosylase reporter assays used in this study. Thymidine is inserted into position 444 in the transcribed strand of the pmax mOrange plasmid (such that it lies opposite dG). This creates a premature stop codon, completely abolishing fluorescence. Removal of thymidine followed by insertion of dC opposite dG restores reporter fluorescence.

### Measurement of ATA In Vivo Using a Host-Cell Reactivation Assay

Human breast cancer MCF-7 cells were seeded at 50,000 cells per well in a 12-well plate, while HAP cells (wild type or UNG knockout) were seeded at 75,000 cells per well in a 6-well plate. Cells were allowed to adhere overnight and then cells in duplicate wells were treated with DMSO or ATA (2.5, 5, or 10 μM) for 24 hr such that the DMSO concentration in each well was 0.1%. Following drug treatment, one well was transfected with the undamaged cocktail and the other well was transfected with the uracil repair cocktail. Transfection was carried out using Lipofectamine 3000 (ThermoFisher) according to the manufacturer's instructions. In brief, each transfection utilized P3000 reagent (4 μL) and Lipofectamine 3000 reagent (3.75 μL) mixed with the plasmid cocktail in serum-free medium (Opti-MEM, ThermoFisher). For experiments in 12-well plates, 100 μL of medium was used, and 200 μL of medium was used for experiments in 6-well plates). After 24 hours, cells were dissociated by trypsinization and analyzed by flow cytometry using an Atune NxT flow cytometer. Gating and compensation were determined by transfection of single-color controls. Reporter expression was calculated for each dose of ATA as previously described and normalized to DMSO treatment.

### Purchased Chemicals

Commercial ATA was purchased from Sigma Aldrich (product #A1895). ATA NH4 salt was purchased from Alfa Asear.

### Further Sigma ATA Chemical Purity Analysis

Purities of final compounds were assessed by analytical reverse-phase HPLC performed with the following method: Dionex UltiMate 3000 series (Thermo Scientific) with a Thermo Scientific Hypersil GOLD C18 (2.1 mm×50 mm, 3 μm particle size) column with the gradient 10-100% ACN/water (10 min), and 100% ACN/water (5 min) flow=0.2 mL/min. The mobile phase was buffered with 0.1% formic acid. Mass spectra were obtained on a Thermo Scientific ISQ EC via negative electrospray ionization. Semipreparative reverse-phase HPLC was performed on am ACCQPrep HP125 system (Teledyne Isco) with a Thermo Scientific Hypersil GOLD C18 (21.2 mm×250 mm, 5 μm particle size) column. NMR spectra were recorded on an Agilent 400 MR instrument and were calibrated using residual undeuterated solvent as an internal reference (DMSO-d6: 1H NMR δ=2.50 ppm, 13C NMR δ=39.52 ppm). The following abbreviations were used to explain NMR peak multiplicities: s=singlet, d=doublet, br=broad. 2-Hydroxybenzoic acid was purchased from TCI (lot: XXHPA-QE). 2,2′-Dihydroxy-5,5′-methylenedibenzoic acid was purchased from Acros (lot: A0387467) and purified by HPLC before use.

5,5′,5″-(hydroxymethanetriyl)tris(2-hydroxybenzoic acid) (1) A powdered mixture consisting of 2,2′-Dihydroxy-5,5′-methylenedibenzoic acid (302 mg, 1.05 mmol) and salicylic acid (145 mg, 1.05 mmol) was added, in portions, to a solution of sodium nitrite (145 mg, 2.10 mmol) in concentrated sulfuric acid (2 mL). The reaction was stirred at room temperature for 24 h. The reaction mixture was added to 100 g of ice in a beaker. Crude precipitate was filtered and washed with water. The residue was purified by semi-preparative HPC to afford pure 1 (73 mg, 16%). The preparation of 1 has been previously reported in the literature.

### Physical State

Red solid; TLC: Rf=0.14 (DCM/MeOH/TFA 95:5:2, UV active); ¹H NMR (400 MHz, DMSO-d6) δ 13.90 (s, 3H), 11.22 (s, 3H), 7.64 (d, J=2.5 Hz, 3H), 7.29 (dd, J=8.7, 2.5 Hz, 3H), 6.91 (d, J=8.7 Hz, 3H), 6.55 (br, 1H). ¹³C NMR (101 MHz, DMSO-d6) δ 171.74, 159.97, 138.25, 135.13, 128.86, 116.61, 111.88, 78.98. MS (ESI−): C22H16O10 [M−H]⁻: 439.23, [M−H2O−H]⁻: 421.09, [M+FA−H]⁻: 485.10, [2M−H]⁻: 879.33.

### Results

### Identification of the Human UDG Inhibitor Aurintricarboxylic Acid

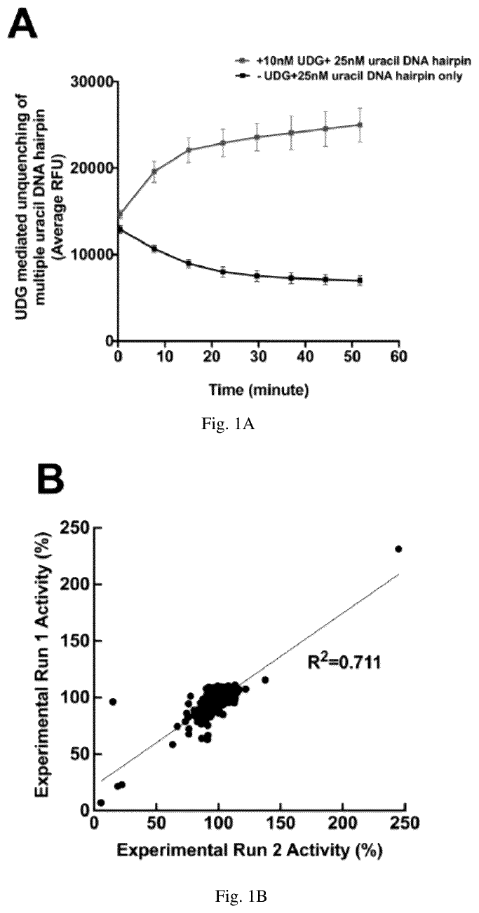

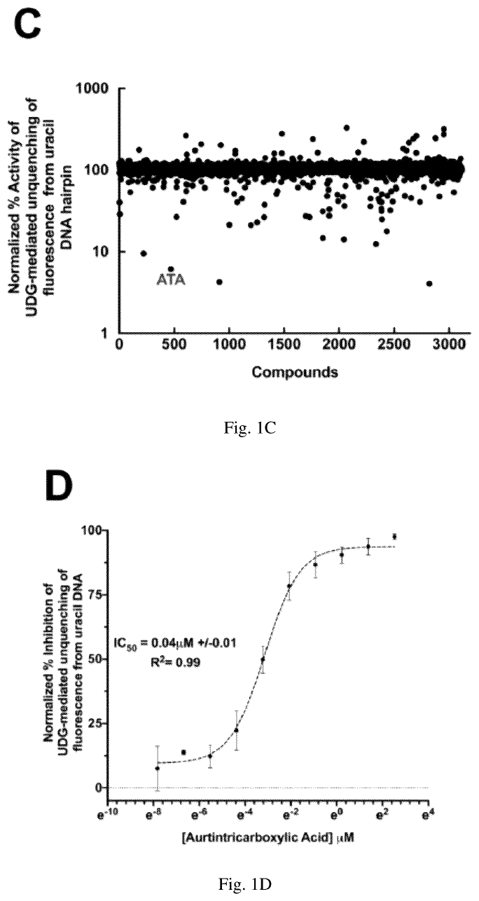

To identify small-molecules that target the human UDG activity, we employed high-throughput screening (HTS) on a focused diversity library. We optimized a homogenous high-throughput screening platform. This assay measures removal of multiple uracils from a DNA hairpin (termed uracil DNA hairpin), resulting in loss of hydrogen bonds between U:A base pairs. Consequent melting of the hairpin results in fluorescence when the 5′ end FAM is separated from the 3′ end quencher dabcyl. A small-molecule that blocks the activity of UDG in the assay leads to retention of some uracils in the DNA hairpin and a reduction in the fluorescence level (RFU, relative fluorescence unit). In the absence of inhibitor, monitoring the reaction with UDG protein over 60 minutes shows an increase in the average RFU of the DNA substrate over time (FIG. 1A). Three hundred twenty small-molecules were screened in two independent experiments. The correlation coefficient (R2) was 0.711 (FIG. 1B). Extensive optimization increased the sensitivity of the assay in a 384-well plate format. We successfully employed this assay with a pharmacologically active 3,114 diversity compound collection, including FDA approved drugs, at a concentration of 12.5 μM (FIG. 1C). The final assay conditions have an average signal-to-noise ratio of 3.5, leading to an average Z prime.

After screening the selected diversity library, we first selected 16 compounds that blocked UDG activity by more than 50%. We then validated these compounds by performing dose-response experiments using the same primary HTS assay in a 384 well plate format. We confirmed the activity of each active compound by serial dilution into the low nanomolar range. We then selected the top bioactive small molecules with a 50% inhibitory concentration (IC50) less than <1.0 μM for further characterization. We eliminated known DNA intercalator drugs and several small-molecules that nonspecifically target uracil-DNA hairpin substrate directly by quenching the residual DNA fluorescence in the absence of UDG protein. We selected aurintricarboxylic acid (ATA) (Sigma Aldrich) as the most potent of the screened compounds with an initial reproducible submicromolar inhibitory constant (IC50) value of 40 nM for the purified human UDG (FIG. 1D). ATA provides a completely different chemotype from uracil so we proceeded to characterize its activity and mechanism.

### Time Course of Human UDG Activity

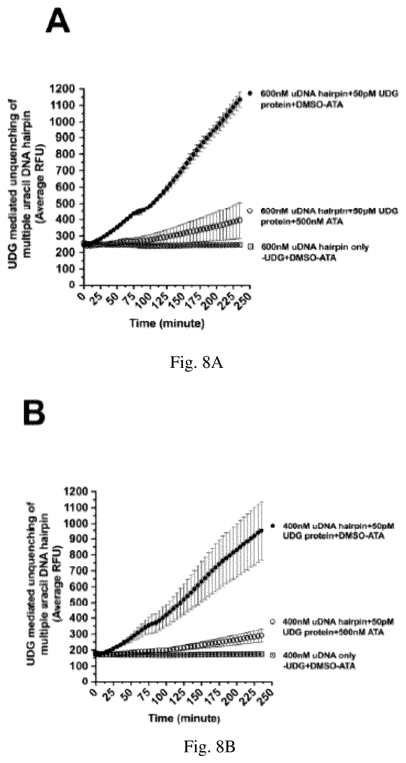

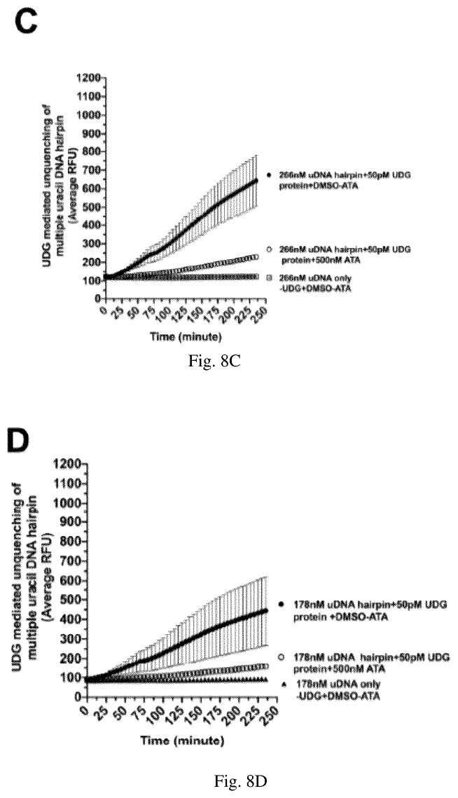

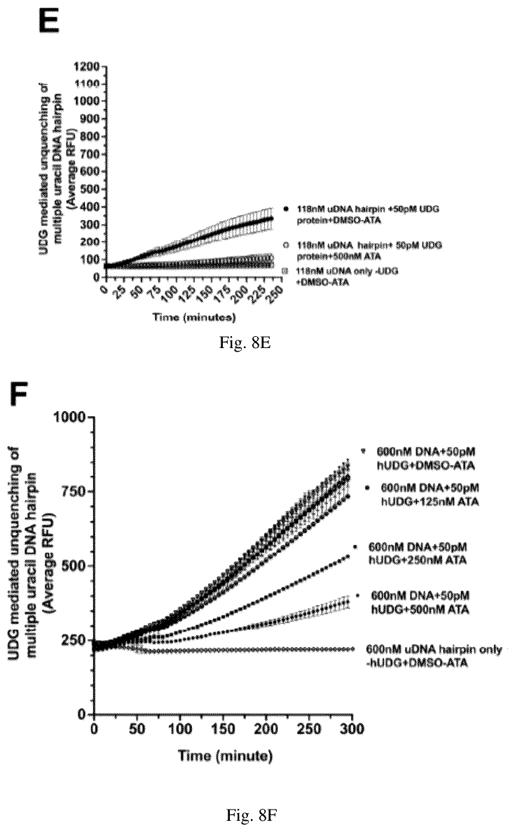

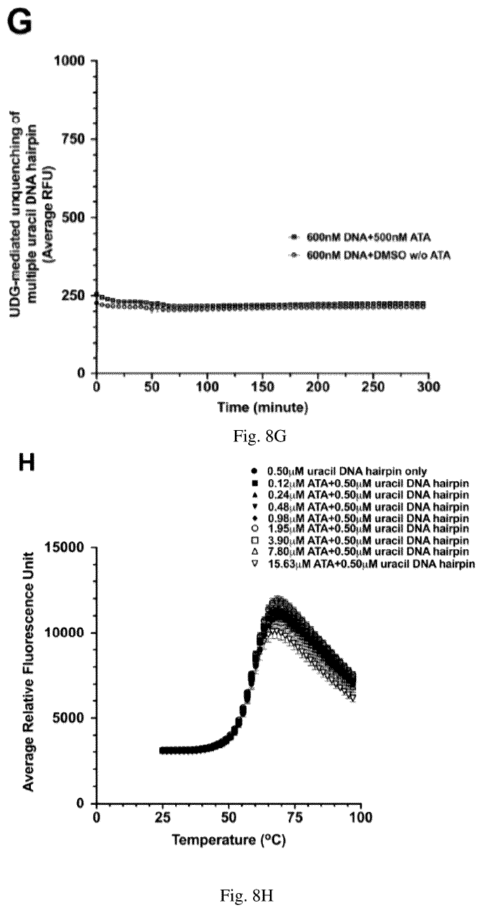

To better examine UDG inhibitor activity, we monitored human UDG activity over time. Time course experiments demonstrated that ATA at a fixed dose of 500 nM persistently diminished UDG activity over a range of uracil DNA hairpin FIGS. 8A-E). This suggests that the compound blocks the interactions between UDG and uracil DNA hairpin in all tested time points. We did not observe ATA interference with the substrate residual fluorescence signal in the absence of UDG protein over the same time course.

We next studied the impact of titrating lower ATA concentrations using the above assay but fixing the amount of DNA substrate. We found that below 250 nM, ATA was unable to significantly compete with excess DNA substrate and reduce UDG-mediated un-quenching of u-DNA hairpin over a 5-hour time course (FIG. 8F). In the absence of UDG protein, ATA has no effect on the basal uracil DNA hairpin fluorescence over the same time course (FIG. 8G).

ATA does not Impact the Melting of Uracil Containing-DNA Hairpin Substrate

Many chemotherapeutic DNA damaging agents are known to target DNA rather than proteins directly. To test if ATA disrupts DNA substrate, we measured the impact of ATA on the thermal unfolding of uracil containing DNA hairpins (U:A base pairs) through FAM fluorescence (directly labeled on the DNA hairpin). We first determined the melting temperature (Tm) of the uracil DNA hairpin in the absence of both UDG protein and ATA compound (DNA-only condition). We then asked whether ATA directly targets the uracil containing DNA substrate in the absence of UDG. We found (FIG. 8H) that ATA did not shift the melting curve (Tm) of uracil containing DNA in the absence of UDG protein. This observation suggests that ATA does not target the uracil containing DNA substrate in the reaction.

The Co-Crystal Structure of UDG with ATA

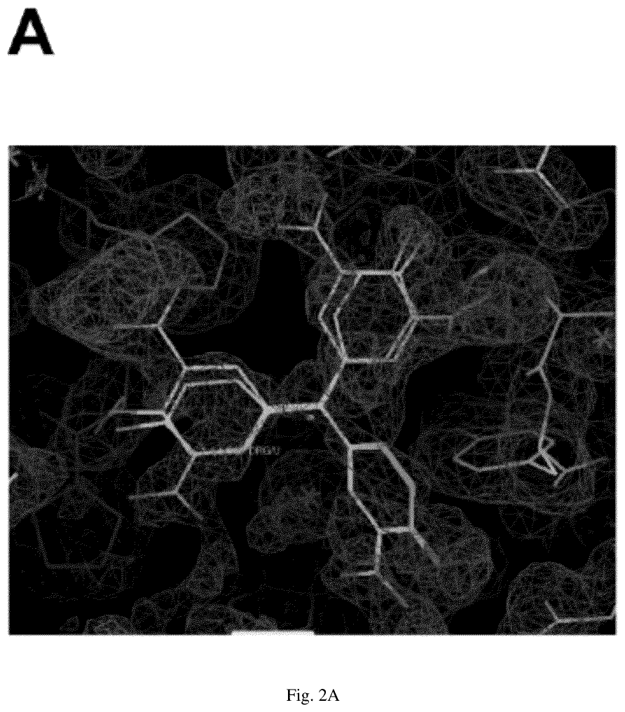

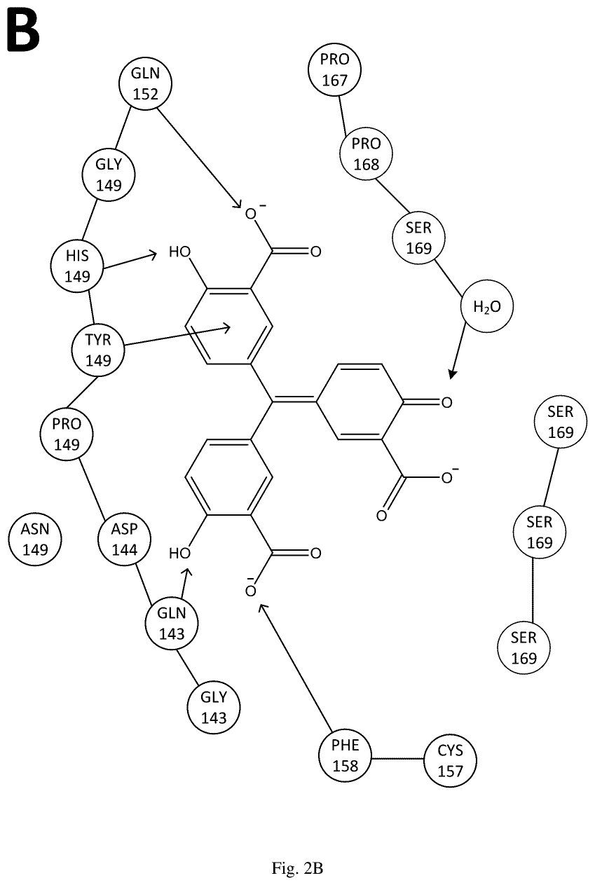

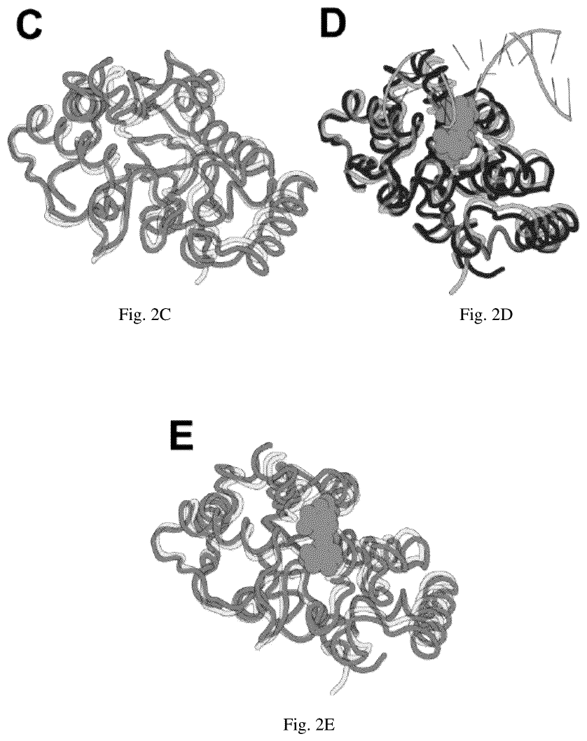

To experimentally examine the ATA interaction site with UDG, we co-crystallized UDG with ATA, collected X-ray diffraction data, and refined the crystal structure to 1.8 Å resolution (Table 1). We find ATA bound to the active site of UDG coordinated by two water molecules (FIG. 2A). The electron density indicates some flexibility, and shows that the three-fold symmetric ATA inhibitor binds with one moiety pointing out from the active site disordered. The two groups point into the active site pocket and bind in two rotamers. Notably, one moiety stacks with Tyr147 and His148, and hydrogen bonds with Gln152. Refinement and difference map analyses supports two inhibitor binding modes of ATA that block uracil access to the active site. Importantly the clear electron density for the protein shows that both ATA binding modes hold UDG in its open conformation effectively blocking the open to closed conformational switch needed to strongly bind DNA and to expose, recognize, and excise an extra-helical uracil (FIG. 2B). Panels of cartoons representing the different UDG conformations are shown in FIG. 2C-2E, unbound UDG open conformation (FIG. 2C), closed DNA bound UDG conformation (FIG. 2D) and ATA bound UDG open conformation (FIG. 2E).

### Computational Analyses of Uracil, ATA and Salicylic Acid Binding to UDG

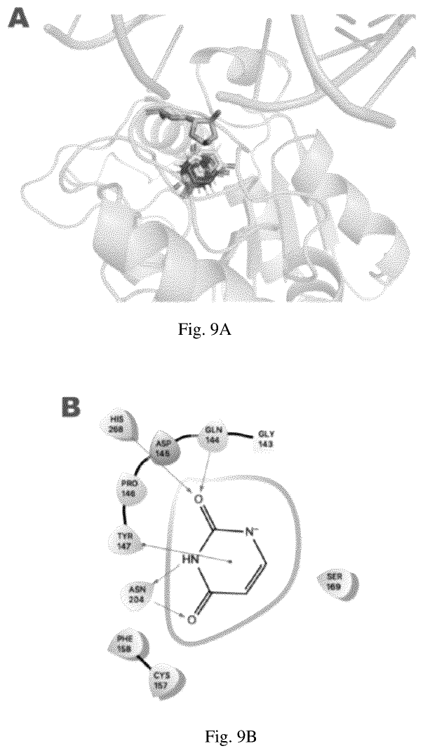

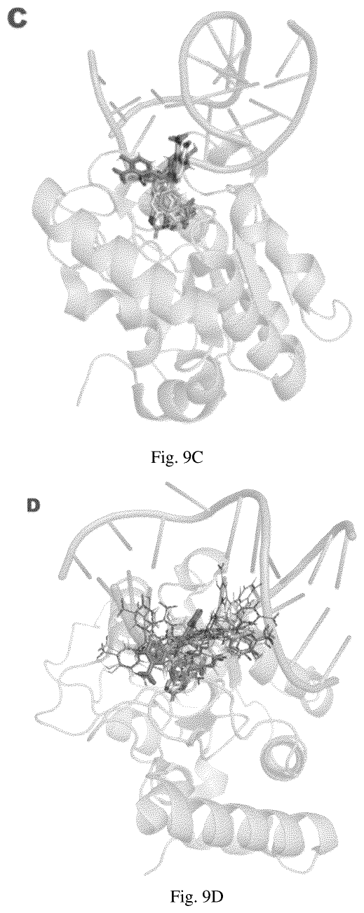

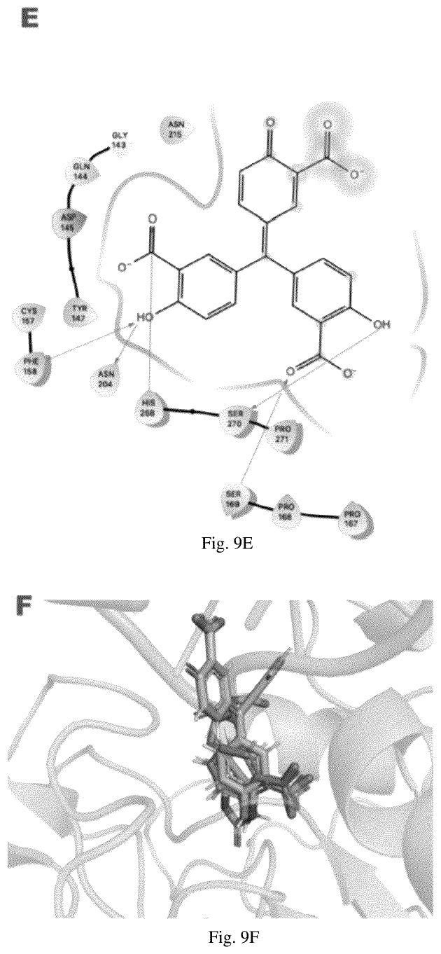

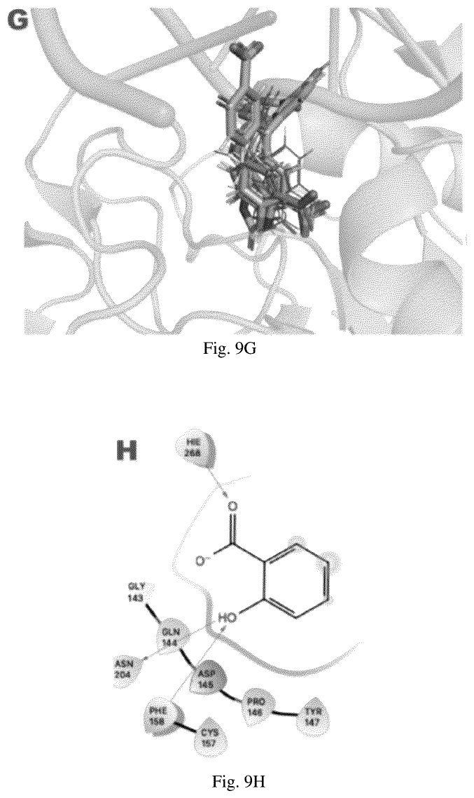

To computationally test ATA binding orientations, we used in silico evaluations of multiple ATA conformations and its composing moieties by docking with Glide (Schrodinger suite for drug discovery). We examined the binding and multiple possible conformations of ATA in complex with UDG for four structures of UDG available in the Protein Data Bank (PDB IDs: 1AKZ,1UGH, 1EMH, and 1SSP). As a control, this docking method suitably identifies the uracil base fit to the uracil ring UDG structures. Importantly, ATA docks into poses consistent with the electron density for the DNA-free UDG conformation. We found a similar agreement for the ATA salicylic acid moiety, which also docks into the uracil binding site. We also examined possible ATA binding to UDG conformations bound to uracil in dsDNA, which shows a more closed active site characterized by inward shifts of residues that coordinate DNA binding, bending, uracil nucleotide flipping from duplex DNA and uracil binding. Interestingly, the docking results of ATA to the dsDNA-bound UDG structure show alteration in one of UDG's β strands, and a shift closing the β strand is part of the open to close transition. These findings are consistent with ATA predominantly binding to UDG alone in its open conformation and thereby blocking the UDG-DNA complex. Collectively these computational results suggest that although ATA could potentially bind to the closed, DNA-bound UDG active site with a β strand shift, the results largely support ATA binding to the open DNA-free UDG (FIG. 9A-I).

### ATA Binds Directly to Purified UDG

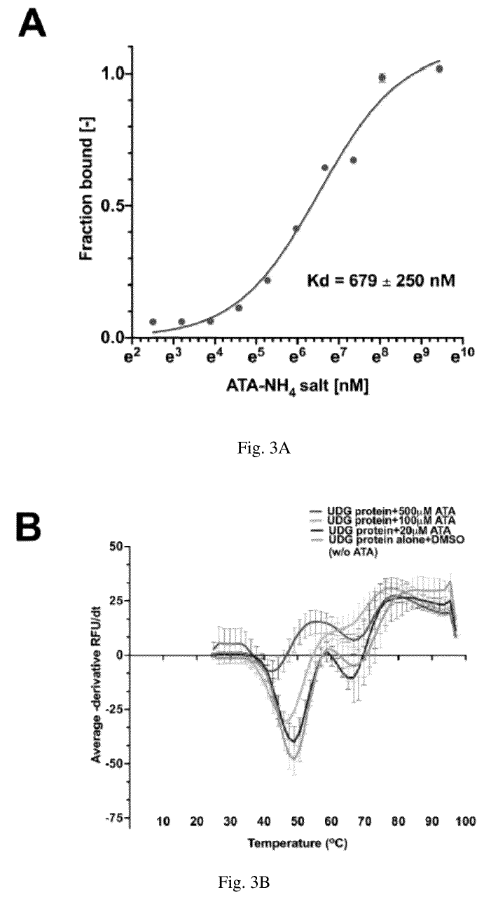

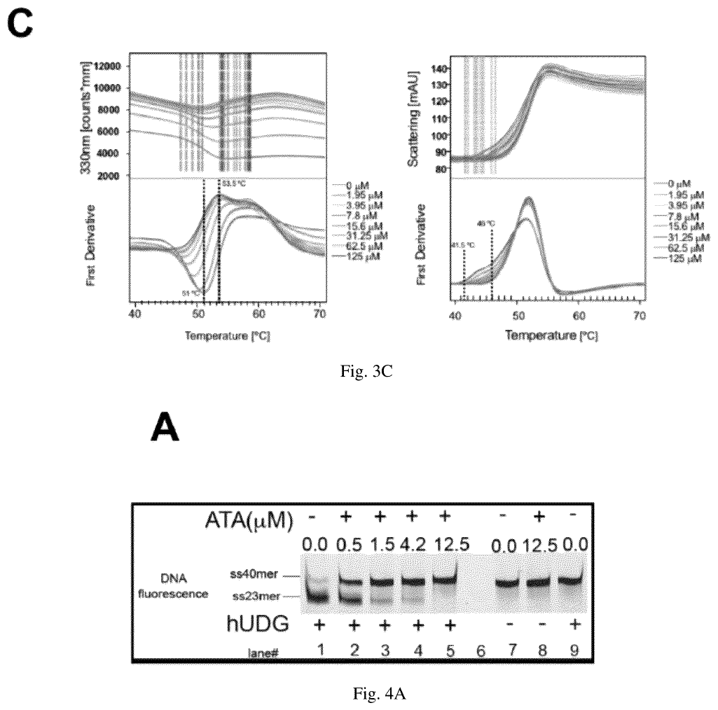

To experimentally test the binding between the human UDG protein and ligand ATA, we employed microscale thermophoresis (MST). The N-terminal primary amine was labeled with Ato488, and 100 nM labelled-UDG titrated against the increasing concentration of ATA. The MST binding isotherms revealed an affinity binding dissociation constant (KD) of the more soluble ATA NH4 salt form displays an estimated KD of <700 nM (FIG. 3A) which resembles the DNA cutting results with an estimated IC50 of 700 nM. Using an independent protein thermal shift assay using fluorescence dye that preferential binds to unfolded protein, we observed ATA dose-dependent destabilization of the human UDG protein (FIG. 3B). To further examine the direct binding of ATA to UDG, we measured the denaturation of the UDG protein as a function of increasing temperature with and without ATA. We used nanoDSF technology to measure the intrinsic tryptophan fluorescence of protein during unfolding. The thermal stability of UDG in the presence of titrating concentrations of ATA was measured. The results showed a decrease in the mean melting temperature of UDG from 53.5° C. to 51° C. in the presence of ATA suggesting that the compound destabilizes UDG (FIG. 3C, left panels). Note that the melting temperature for UDG with 0 μM ATA is at 53.5° C. shown by a dotted line on the first derivative peak corresponding to the red lines on the 330 nm panel. While generally thermal stability or instability observed by a shift in the peak melting temperature, ATA binding results in protein aggregate formation at lower temperatures, which associate with surface exposed tryptophan resulting in fluorescence quenching at the 330 nm channel. This resulted in downward shift of the melt curve (first derivative melting temperature peak change from upwards to downward direction) oppose to a peak-shift. This is further confirmed by the scattering data, which marks the beginning of protein denaturation where UDG in the presence of ATA clearly induced protein aggregation at lower temperatures than UDG alone (FIG. 3C, right panels). Though, we could not derive a binding constant using the protein denaturation assays, our results verify the direct binding of ATA to the human UDG protein. Based on our results, we reasoned ATA binding maintains UDG in an open conformation that loses a main chain β strand hydrogen bond and the more closed active site.

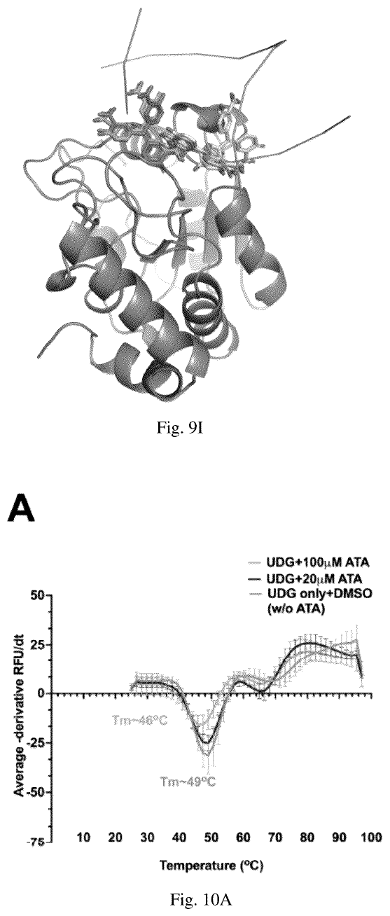

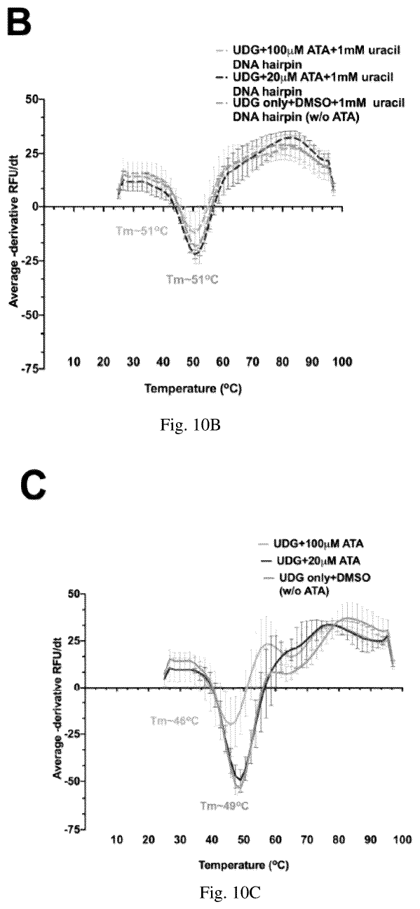

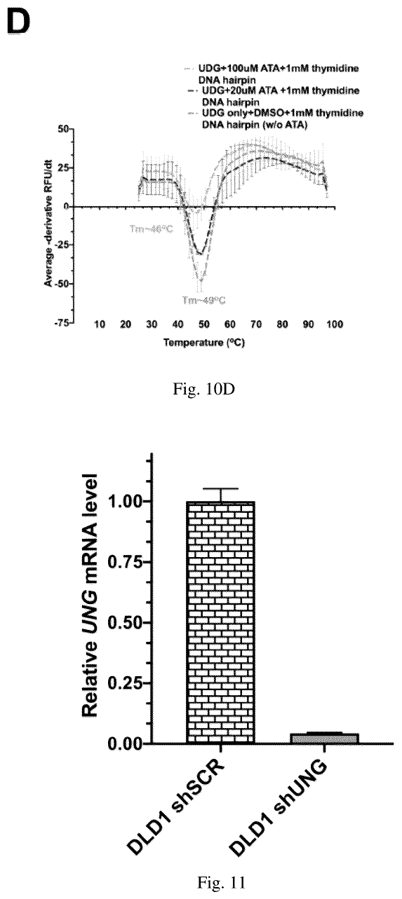

We also tested whether the addition of uracil containing DNA would alter the ATA associated decrease in UDG Tm. In the absence of uracil containing DNA, an average maximum Tm change for UDG in the presence of 100 μM ATA is ˜3.0° C. FIG. 10A. However, with the addition of 1.0 mM of uracil DNA hairpin, UDG was stabilized, and the addition of 100 μM of ATA did not further change the Tm of UDG protein (FIG. 3B). The results suggest that excess uracil containing-DNA could stabilize the UDG protein consistent with the hydrogen-deuterium exchange results that establish an extended dsDNA binding region outside of the active site pocket that may complete with the ATA.

In contrast, excess normal thymidine containing DNA hairpin did not stabilize the UDG protein even at millimolar concentration (FIGS. 10C-D). Together these results show that UDG stabilization depended upon direct binding to uracil DNA hairpin and that ATA binds to and destabilizes the open, DNA-free UDG conformation.

### The Efficiency of ATA Inhibition of Human UDG

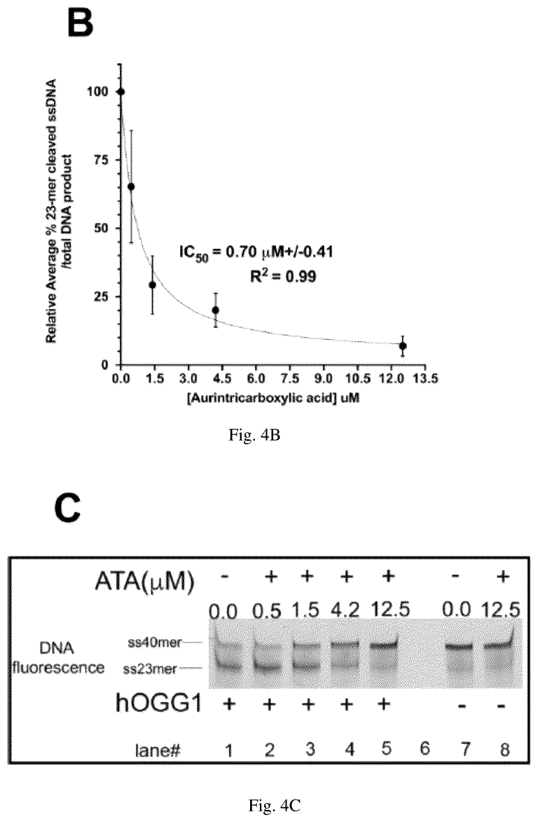

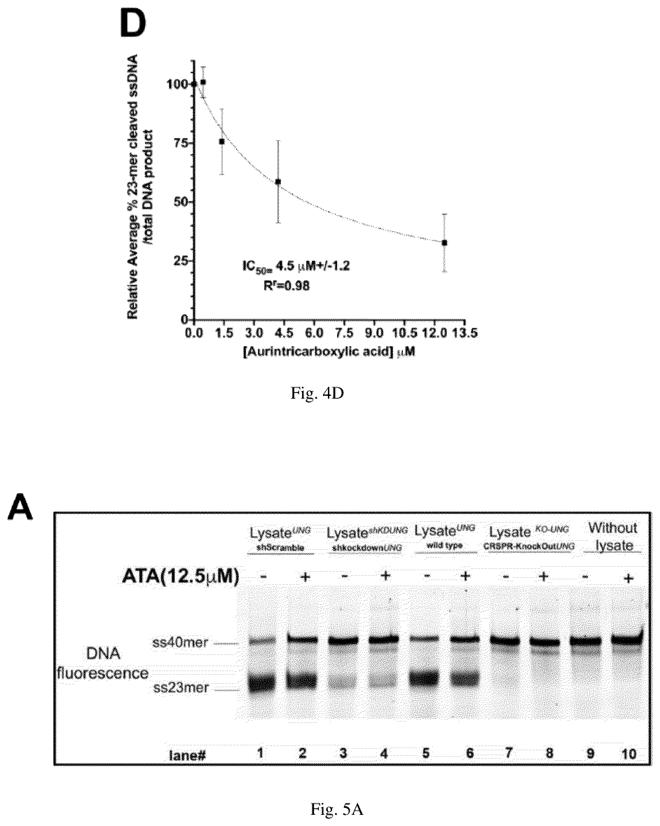

To test ATA efficiency for UDG inhibition, we employed a dsDNA oligo containing single U:A base pair and examined heat sensitive DNA cleavage on a gel-based assay. We compared the specificity of ATA inhibition for UDG acting on uracil-containing DNA with inhibition of 8-oxoguanine DNA glycosylase (OGG1) acting on dsDNA containing a single 8-oxoguanine paired with cytosine (8oxG:C bp). In this assay, ATA demonstrated an average IC50 value of 0.70 μM+/−0.41 towards UDG, R²=0.99) (FIG. 4A-B). In contrast, ATA exhibited a lower inhibitory effect on human OGG1 protein with an average IC50 of 4.5 μM+/−1.2 μM R²=0.95) (FIG. 4C-D), showing a 5-fold difference and demonstrating a more modest ATA inhibitory effect on UDG over OGG1, despite the recognized cross-reactivity of ATA with nucleotide binding enzymes.

### ATA is an Effective Inhibitor of UDG in Human DLD1 Colon Cancer Cell-Extracts

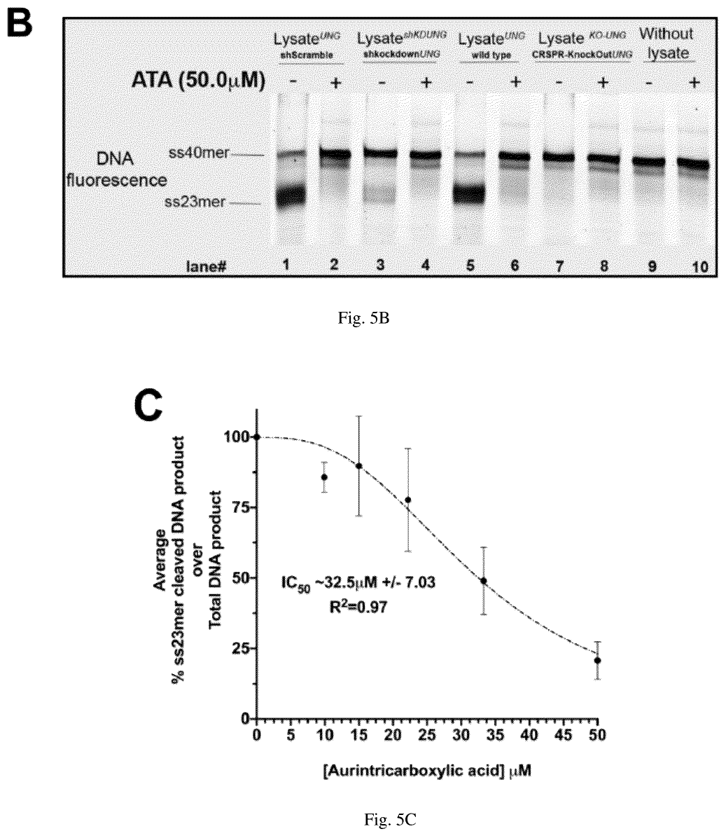

To test possible cell relevant biological activity, we conducted UDG inhibition assays with whole cell extracts. As a control and to further test the efficiency of UDG over other glycosylases, we generated UNG knockdown (shUNG) and CRISPR-Cas9 knockout (UNG-KO) clones in DLD1 human colon cancer cells (FIG. 11) showing shSCR and shUNG knockdown mRNA levels. We performed an in vitro DNA cleavage assay using the uracil containing DNA oligo as described above (FIG. 4). Cell-extracts from the scrambled shRNA and wild type expressing DLD1 cells were used as controls, and UDG-mediated 23mer ssDNA products were readily detectable (FIG. 5A lanes 1 and 5). Cell-extracts derived from UDG knockdown cells, however, showed a drastic reduction in cleavage products (FIG. 5A, lane 3), and the UDG-KO cell extracts showed a complete lack of the ss23mer DNA products (FIG. 5A, lane 7). Treatment with 12.5 μM of ATA, has minimal effect on the 23mer ssDNA generation (FIG. 5A, lanes 2, 4, 6, and 8). However, 50 μM of ATA caused near-complete inhibition of 23mer ssDNA appearance in all experimental conditions where UDG was present (scramble shRNA and WT cell extracts, FIG. 5B, lane 2, 4, and 6). Notably, UNG-KO cell-lysate failed to generate the ss23mer DNA products regardless of the ATA concentrations (FIGS. 5A and 5B, KO UNG lanes). Thus, our consolidated results revealed that the 23mer ssDNA product could be attributed to UDG activity. FIGS. 4A and 4B, lane 10 (a control) shows that in the absence of UDG and cell lysate, ATA even at a concentration of 50 μM alone does not dampen fluorescence signal of the DNA oligo in the reaction and the compound does not generate secondary non-specific cleaved DNA bands. This further suggests that ATA does not disrupt the DNA substrate.

We then carried out dose-response experiments, titrating lower concentrations of ATA. We estimated the IC50 in DLD1 UDG containing cell lysate which is approximately 32.5 μM+/−7.03 (FIG. 5C), consistent with ATA having affinity for other macromolecules in the cell lysate. Overall, these data provide biologic evidence of ATA inhibitory efficacy towards UDG in disrupted cells. A knockout of UDG or pharmacological inhibition with ATA completely inhibited the generation of the ss23mer UDG enzymatic products (FIG. 5B), demonstrating empirical specificity of our UDGi for its target.

### ATA Inhibition of Uracil Removal in Human Cell Lines

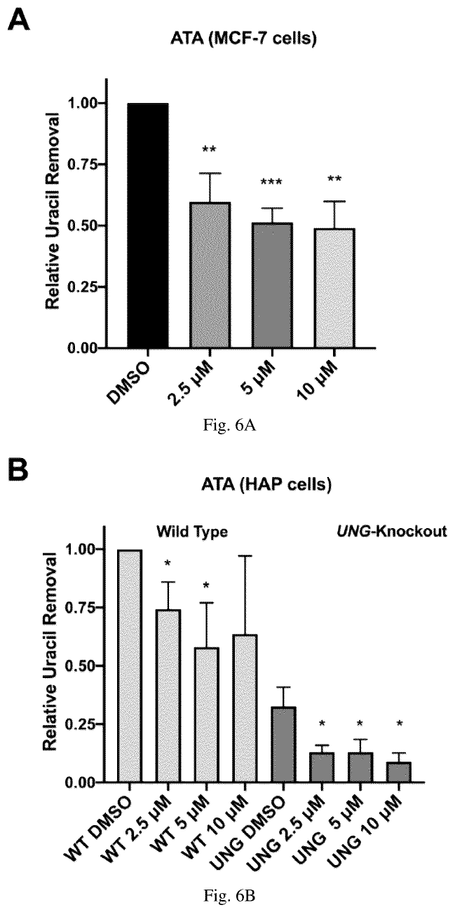

We used a plasmid-based host cell reactivation assay to measure uracil removal from a U:G base pair in human cell lines. Treatment with ATA (2.5 μM significantly inhibited uracil removal in MCF-7 cells (FIG. 6A). Increasing concentrations of ATA up to 10 μM were not statistically different from the 2.5 μM dose. We next measured the effectiveness of ATA for UDG inhibition by measuring uracil removal in wild type and UDG-knockout HAP cells (FIG. 6B). Knockout of UDG reduced uracil repair by approximately 3-fold. Interestingly, UDG-knockout cells removed uracil from at least 99% of plasmids before fluorescent protein could be expressed, (flow cytometry Figure/table), suggesting that backup uracil removal pathways operate with high efficiency. ATA (5.0 μM) reduced uracil removal in wild type cells by 1.8-fold, accounting for 60% of the effect of UDG knockout (FIG. 6B). ATA (5 μM) reduced relative uracil removal in UDG-knockout cells from approximately ˜0.3 to ˜0.1. We cannot quantify the contribution of glycosylases such as SMUG1 to uracil repair in UDG-knockout cells, so the extent to which ATA inhibits these enzymes cannot be determined in this assay. However, the data suggest that inhibition of uracil removal by ATA in wild type cells is predominantly due to UDG inhibition, consistent with its structural interactions with the active site pocket noted above.

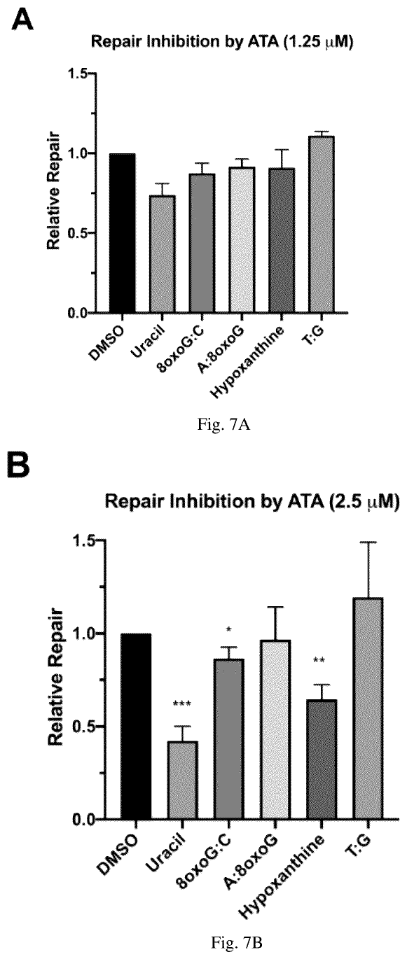

We also compared the inhibitory effects of ATA on the repair of other glycosylase reporter substrates 8oxoG:C (OGG1), A:8oxoG (MPG, MUTYH), hypoxanthine:T (MPG), and T:G basepairs (TDG, MBD4) in MCF-7 cells, FIG. 7A-B). We found that ATA (2.5 μM) inhibits uracil repair (0.58 reduction in relative repair of uracil, FIG. 7B) significantly more than repair of other transfected reporter plasmids (0.13 reduction in relative repair of 8oxoG:C and 0.35 reduction in relative repair of hypoxanthine:T repair while not inhibiting the repair of a T:G or A:8oxoG basepair, FIG. 7B).

A primary task for preclinical investigation of BER as a potential cancer therapy target is to develop chemical inhibitor tools that target appropriate BER enzymes in cells. We reasoned that detection and removal of the damaged bases is the crucial committed step in initiating BER and thus in defining DNA base repair outcomes. Furthermore, subsequent BER steps are likely to proceed by DNA product handoffs to avoid the release of toxic and mutagenic intermediates, and such dsDNA handoffs between proteins may prove more challenging to completely block by small molecule inhibitors. We therefore focused on the prototypic DNA glycosylase UDG, which removes uracil resulting from cytosine deamination or uracil misincorporation. Moreover, cancer cells expressing high levels of the constitutively nuclear single-stranded DNA deaminase APOBEC3B and functional p53 can be killed by a UDG protein inhibitor, resulting in a recognized need to develop effective small molecule inhibitors to UDG.

These considerations support the notion that inhibiting UDG and other BER glycosylases may prove advantageous over other BER enzymes for cancer biology and prompted our focus on UDG inhibitors. Here we report the development and application of an assay to screen chemical libraries for UDG inhibitors resulting in identification and characterization of the exemplary UDGi ATA, which is effective in human cancer cell extracts and for uracil containing reporter plasmids in cancer cells. A current successful structure-based inhibitor strategy is to screen crystals with chemical fragment libraries and then use resulting protein-fragment X-ray crystal structures to design more specific inhibitors. Our ATA results support the notion that we can harness the general ATA ability for nucleotide mimicry to empirically employ it as a UDG inhibitor tool that furthermore informed us on the strategy and chemistry to effectively inhibit the glycosylase.

First, by identifying ATA from an unbiased chemical diversity screen and examining its binding to UDG by crystallography and computational docking, we gain insights for a different conceptual approach for developing inhibitors to BER glycosylase enzymes as exemplified by UDG. Second, our structural and computational results suggest a novel strategy to inhibit UDG by targeting the open catalytically inactive conformation that is both catalytically incompetent and not only less able to bind DNA but also less stable providing a possible dual means to reduce UDG activity in cells as consistent with significant activity in cell extracts and cells. Third, the ATA bound structure and assays provide a chemical framework distinct from uracil substrate, which allows the closed DNA binding conformation, for further optimization by synthetic organic chemistry.

Transition-state theory encompassing “lock and key” substrate recognition and high-affinity binding to transition states have provided a robust conceptual framework for examining enzyme action. So transition-state approaches been used for designing new enzymes from proteins, including catalytic antibodies. Furthermore, for many enzymes, inhibitors are deliberately designed as transition-state analogs.

In addition, inhibitor design has used the concept of anchored plasticity where binding to a stable site is used to extend inhibitor chemistry and push open adjoining regions to gain specificity, as successfully done for inhibitors for nitric oxide synthetase MRE11 nuclease, and poly(ADP-ribose) glycohydrolase (PARG). Yet, to strategically develop inhibitors that target DNA glycosylases and BER for preclinical cancer studies, our ATA results here suggest that it may not be enough to catalog what these repair enzymes do and find possible inhibitors; we may also need to consider how the enzymes and inhibitors act.

We find that ATA does not disrupt uracil-containing DNA in vitro, and it directly binds to human UDG showing that ATA is working by binding free UDG prior to its DNA binding. UDG recognition of uracil in DNA is a cooperative phenomenon involving multiple individual binding interactions of UDG with both DNA backbone and uracil nucleotide that enable the precise fit of the catalytically competent UDG-DNA substrate complex. Importantly, these interactions are synergistic and include conformational closing of both the DNA binding channel and the uracil binding pocket. By strategically interfering with some of these interactions and with the conformational closing required for specific uracil recognition and excision, ATA constitutes an effective UDGi despite having multiple bound conformations, not resembling a transition state analog and not pushing open adjoining sites. Instead, ATA binds to the open active site, makes interactions with some key residues for uracil binding, and resists conformational closing that is the cooperative mechanism for uracil recognition and excision. Strikingly, this is the same strategy as that employed by the viral Ugi protein inhibitor, which also binds to the open catalytically incompetent conformation of UDG. This structurally-supported concept of binding the open state and blocking the conformational closing needed for substrate recognition explains our collective data on inhibition, binding, and lowering the UDG melting temperature. Importantly, novel synthesized molecules based upon ATA, chemically distinct from the natural uracil-DNA substrate, will likely similarly hold the UDG active site open to effectively block uracil recognition and removal. Interestingly the open conformation bound by ATA is not in the DNA binding conformation and also has less stability. This may come in part from the loss of a beta-strand hydrogen bond as seen in our new structure that matches the prior DNA-free UDG structure. This finding of destabilization suggests the notion that an optimal small-molecule would not only inhibit UDG but furthermore reduce its stability to effectively reduce its activity in cells by both inhibition and destabilization. Although we identified ATA with a fluorescence screen, it may be possible to employ biophysical techniques such as X-ray scattering to screen directly for binding to a specific conformational state.

Overall, the results from our inhibitor screen and characterization of ATA as a UDGi teach us fundamental strategies for nucleic acid research and cancer. The small molecule need not compete with UDG binding to DNA. Instead, compounds that effectively hold UDG in the open (catalytically incompetent) and less stable conformation precluding damaged DNA substrate recognition and base removal may be equally or even more effective. This may provide a critical enabling strategy for UDG as results from both hydrogen-deuterium exchange and computational docking show that the DNA binding area for UDG (and by implication other BER enzymes) is more significant than indicated in crystal structures suggesting it may be quite challenging to block with a small-molecule chemical inhibitor. Yet as a “door stopper” ATA does not need to compete with DNA; it only needs to block UDG closing and thus prevent the complementary DNA binding channel and the lock-and-key specific substrate recognition. In this analogy, the preclinical UDG inhibitor ATA need not have key-like specificity: instead, it needs to provides a non-uracil chemotype suitable for chemical optimization to improve affinity and specificity to the open UDG conformation. In this inhibitor design strategy the ATA chemotype is a critical enabling tool because, unlike uracil and transition state chemotypes that would in fact promote the formation of the complementary DNA binding channel and active site closing, ATA blocks this closure and hence blocks strong DNA binding and the chemically functional conformation. Notably, other DNA base and nucleotide excision enzymes such as MutY, APE1, FEN1, MRE11, WRN and XPG employ an open-to-closed conformational switch to create the catalytically competent active site only for their specific substrates. We therefore propose that the “door stopper” inhibitor strategy identified here by the UDG-ATA complex can be used as a potential general strategy to block excision repair enzymes for cancer biology. Currently these findings provide proof-of-principle for development of the ATA chemotype and “door stopper” strategy targeting inhibitor binding to an open pre-catalytic glycosylase conformation and preventing active site closing for functional binding needed to excise damaged bases in DNA.

Indeed, ATA effectively inhibits UDG-mediated cleavage of uracil-containing DNA in human DLD1 colon cancer cell extracts and in human cell lines. These observed reductions of UDG activity in human cancer cell lysates and cells suggests that an ATA-based inhibitor strategy may effectively block intracellular UDG activity. Furthermore, the assay for un-quenching of fluorescence from the DNA hairpin with its initial calculated 40 nM IC50 was further validated with a single U containing oligo of dsDNA in a DNA cutting assay supporting an IC50 in the submicromolar range (˜0.7 μM). Binding data with the more soluble form of ATA (ATA NH4 salt) shows significant improvement in direct binding to the purified human UDG protein. The structural and docking results show that by binding the open UDG conformation ATA decrease stable DNA binding and blocks uracil flipping and excision.

Taken together, our findings identify ATA as a compound able to inhibit UDG by holding the protein in the open conformation, like a door stopper, that can effectively inhibit UDG in cells and cell extracts despite the presence of large amounts of potential non-specific DNA binding.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, we claim:

1. A method of inhibiting uracil-DNA glycosylase in a cancer cell, the method comprising:
selecting an agent that binds to a UDG such that the UDG is maintained in a destabilized, open precatalytic glycosylase conformation that prevents active site closing for functional DNA binding and nuclease flipping needed to excise damaged bases binding in DNA; and
administering the selected agent to the cancer cells.

2. The method of claim 1, wherein agent has a Kd of <700 nM and an IC₅₀ of less than <700 nM for UDG.

3. The method of claim 1, wherein the agent binds free UDG prior to DNA binding.

4. The method of claim 1, wherein the agent promotes destabilization of UDG.

5. The method of claim 1, wherein the agent has a non-uracil chemotype.

6. The method of claim 1, wherein the agent is aurintricarboxylic acid (ATA), an analog or chemotype thereof, or a pharmaceutically acceptable salt, tautomer, or solvate thereof.

7. The method of claim 1, further comprising administering at least one of folate antimetabolite or pyrimidine analog to the cancer cell.

8. The method of claim 1, further comprising administering at least one of pemetrexed, 5-FdU, or 5-FU to the cancer cell.

9. A method of treating cancer in a subject in need thereof, the method comprising:
administering to the subject a therapeutically effective amount of an agent that inhibits uracil-DNA glycosylase, wherein the agent binds to UDG such that the UDG is maintained in a destabilized, open precatalytic glycosylase conformation that prevents active site closing for functional DNA binding and nuclease flipping needed to excise damaged bases binding in DNA.

10. The method of claim 9, wherein agent has a Kd of <700 nM and an IC₅₀ of less than <700 nM for UDG.

11. The method of claim 9, wherein the agent binds free UDG prior to DNA binding.

12. The method of claim 9, wherein the agent promotes destabilization of UDG.

13. The method of claim 9, wherein the agent has a non-uracil chemotype.

14. The method of claim 9, wherein the agent is aurintricarboxylic acid (ATA), an analog, derivative, or chemotype thereof, or a pharmaceutically acceptable salt, tautomer, or solvate thereof.

15. The method of claim 9, further comprising administering at least one of a folate antimetabolite or pyrimidine analog in combination with the agent.

16. The method of claim 15, wherein the folate antimetabolite or pyrimidine comprise at least one of pemetrexed, 5-FdU, or 5-FU.

17. A method of treating cancer in a subject in need thereof, the method comprising:
administering to the subject therapeutically effective amounts of at least one of a folate antimetabolite or pyrimidine analog in combination with an agent that inhibits uracil-DNA glycosylase, wherein the agent binds to UDG such that the UDG is maintained in a destabilized, open precatalytic glycosylase conformation that prevents active site closing for functional DNA binding and nuclease flipping needed to excise damaged bases binding in DNA.

18. The method of claim 17, wherein the agent is aurintricarboxylic acid (ATA), an analog, derivative, or chemotype thereof, or a pharmaceutically acceptable salt, tautomer, or solvate thereof.

19. The method of claim 18, wherein the folate antimetabolite or pyrimidine comprise at least one of pemetrexed, 5-FdU, or 5-FU.
